(19) United States
(12) Patent Application Publication
(10) Pub. No.: US 2023/0334263 A1
(43) Pub. Date: Oct. 19, 2023

(54) AUTOMATING FOLLOW-UP ACTIONS FROM CONVERSATIONS

(71) Applicant: Abridge AI, Inc., Pittsburgh, PA (US)

(72) Inventors: Sandeep Konam, Pittsburgh, PA (US); Shivdev Rao, Pittsburgh, PA (US)

(73) Assignee: Abridge AI, Inc., Pittsburgh, PA (US)

(21) Appl. No.: 18/134,090

(22) Filed: Apr. 13, 2023

Related U.S. Application Data
(60) Provisional application No. 63/330,586, filed on Apr. 13, 2022.

(51) Int. Cl. G06F 40/40; G06F 40/247; G06F 16/34

(52) U.S. Cl. CPC G06F 40/40; G06F 40/247; G06F 16/345

(57) ABSTRACT

Automating follow-up actions from conversations may be provided by analyzing a transcript of a conversation, by a Natural Language Processing (NLP) system, to generate a summary of the conversation in a human-readable format, the summary including action items associated with an identified entity; retrieving, by the NLP system from a supplemental data source, supplemental data associated with the action item that are lacking in the transcript; generating, by the NLP system, a machine-readable message based on the action item and the supplemental data; and transmitting the machine-readable message to a system associated with the identified entity.

### CROSS-REFERENCES TO RELATED APPLICATIONS

The present disclosure claims priority to U.S. Provisional Patent Application No. 63/330,586 filed on Apr. 13, 2022 with the title “AUTOMATING FOLLOW-UP ACTIONS FROM CONVERSATIONS”, which is incorporated herein by reference in its entirety

### BACKGROUND

Many industries are driven by spoken conversations between parties. However, participants of these spoken conversations often mishear, forget, or misremember elements of these conversations, in addition to missing the importance of various elements within the conversation, which can lead to sub-optimal outcomes for the one or both parties. Additionally, some parties to these conversations may need to update charts, notes, or other records after having the conversations, which can be time consuming and subject to mishearing, forgetting, and misremembering the elements of the conversations, which can exacerbate any difficulties in recalling the correct details of the spoken conversation and taking appropriate follow-up actions.

The field of Natural Language Processing (NLP) is a branch of Artificial Intelligence (AI) directed to the understanding of freeform text and spoke words by computing systems. Human speech, despite various grammatical rules, is generally unstructured, as there are myriad ways for a human to express one concept using natural language. Accordingly, processing human speech into a structured format usable by computing systems is a complex task for NLP systems to perform, and one that calls for great accuracy in the output for the NLP systems to be trusted by human users for sensitive tasks.

### SUMMARY

The present disclosure is generally related to Artificial Intelligence (AI) and User Interface (UI) design and implementation in conjunction with transcripts of spoken natural language conversations.

The present disclosure provides methods and apparatuses (including systems and computer-readable storage media) to interact with various Machine Learning Models (MLM) trained to convert spoken utterances to written transcripts and summaries of those transcripts as part of a Natural Language Processing (NLP) system. Various action items can be identified for different parties to the conversation from the transcript (and non-party entities), which differ based on the role of the party in the conversation. The MLMs supplement the data identified from the conversation with data from supplemental data sources that may be used to contextually fill in missing information from the conversation. The MLMs then create machine-readable messages from the unstructured human speech and supplemental data, which can be presented to a user for approval or automatically be sent to a remote system for performing an action item on behalf of the user. These action item outputs are provided in conjunction with one or more of the summary and the transcript via various UIs. As the human users interact with the UI, some or all of the operations of the MLM are exposed to the users, which provides the users with greater control over retraining or updating the NLP system for specific use cases, greater confidence in the accuracy of the underlying MLMs, and expanded functionalities for using the data output by the NLP system. Accordingly, portions of the present disclosure are generally directed to increasing and improving the functionality, efficiency, and usability of the underlying computing systems and MLMs via the various methods and apparatuses described herein via an improved UI.

One embodiment of the present disclosure is a method of performing operations, a system including a processor and a memory that includes instructions that when executed by the processor performs operations, or a computer readable storage device that including instructions that when executed by a processor perform operations, wherein the operations comprise: analyzing a transcript of a conversation, by a Natural Language Processing (NLP) system, to generate a summary of the conversation in a human-readable format, the summary including action items associated with an identified entity; retrieving, by the NLP system from a supplemental data source, supplemental data associated with the action item that are lacking in the transcript; generating, by the NLP system, a machine-readable message based on the action item and the supplemental data; and transmitting the machine-readable message to a system associated with the identified entity.

One embodiment of the present disclosure is a method of performing operations, a system including a processor and a memory that includes instructions that when executed by the processor performs operations, or a computer readable storage device that including instructions that when executed by a processor perform operations, wherein the operations comprise: transmitting, to a Natural Language Processing (NLP) system, audio from a conversation including utterances from a first entity and a second entity; outputting, to the first entity, a first action item assigned to the first entity according to a transcript of the NLP system generated from the audio; receiving supplemental data from the first entity associated with the action item; generating a second action item for an identified entity identified in at least one of the first action item and the supplemental data based on the first action item and the supplemental data; and transmitting a machine-readable message to a system associated with the identified entity.

One embodiment of the present disclosure is a method of performing operations, a system including a processor and a memory that includes instructions that when executed by the processor performs operations, or a computer readable storage device that including instructions that when executed by a processor perform operations, wherein the operations comprise: receiving, from a Natural Language Processing (NLP) system, a transcript of a conversation between at least a first entity and a second entity and a summary of the transcript that includes an action item identified for the first entity to perform; generating a display on a user interface that includes the transcript and the action item; and in response to receiving a selection of the action item from the first entity, adjusting display of the user interface to display a section of the transcript used by the NLP system to identify the action item and an indicator of a supplemental data source used by the NLP system to add additional information to the action item that was not present in the transcript.

### BRIEF DESCRIPTION OF THE DRAWINGS

The accompanying figures depict various elements of the one or more embodiments of the present disclosure, and are not considered limiting of the scope of the present disclosure.

In the Figures, some elements may be shown not to scale with other elements so as to more clearly show the details. Additionally, like reference numbers are used, where possible, to indicate like elements throughout the several Figures.

It is contemplated that elements and features of one embodiment may be beneficially incorporated in the other embodiments without further recitation or illustration. For example, as the Figures may show alternative views and time periods, various elements shown in a first Figure may be omitted from the illustration shown in a second Figure without disclaiming the inclusion of those elements in the embodiments illustrated or discussed in relation to the second Figure.

FIG. 1 illustrates an example environment in which a conversation is taking place, according to embodiments of the present disclosure.

FIG. 2 illustrates a computing environment, according to embodiments of the present disclosure.

FIG. 3 illustrates an action-item creator, according to embodiments of the present disclosure.

FIGS. 4A-4G illustrate interactions with a User Interface (UI) that displays a transcript and action items identified from a conversation, according to embodiments of the present disclosure.

FIG. 5 is a flowchart of a method for presenting action items extracted from a conversation, according to embodiments of the present disclosure

FIG. 6 is a flowchart of a method for using a Natural Language Processing (NLP) system to generate a transcript and action items, according to embodiments of the present disclosure.

FIG. 7 is a flowchart of a method for automating action item extraction and performance using transcripts of natural language conversation, according to embodiments of the present disclosure.

FIG. 8 is a flowchart of a method for displaying transcripts and action items, according to embodiments of the present disclosure.

FIG. 9 illustrates physical components of a computing device, according to embodiments of the present disclosure.

### DETAILED DESCRIPTION

Because transcripts of spoken conversations are becoming increasingly important in a variety of fields, the accuracy of those transcripts and the interpreted elements extracted from those transcripts is also increasing in importance. Accordingly, accuracy in the transcript affects the accuracy in the later analyses, and greater accuracy in transcription and analysis improves the usefulness of the underlying systems used to generate the transcript and analyses thereof.

To create these transcripts and the analyses thereof, the present disclosure describes a Natural Language Processing (NLP) system. As used herein, NLP is the technical field for the interaction between computing devices and unstructured human language for the computing devices to be able to “understand” the contents of the conversation and react accordingly. An NLP system may be divided into a Speech Recognition (SR) system, that generates a transcript from a spoken conversation, and an analysis system, that extracts additional information from the written record. In various embodiments, the NLP system may use separate Machine Learning Models (MLMs) for each of the SR system and the analysis system, or may use one MLM handles both the SR tasks and the analysis tasks.

One element extracted from a transcript can be a follow-up action item. Extracting an action item from a transcript can include determining the identification of a party to perform the action and an identity of the action to perform. As natural human conversations often include implicit assumptions of the knowledge of the participants, references to previously mentioned concepts (e.g., via pronouns, determiners, allusions), different terms used for the same concept (e.g., synonyms, restatements), inferences to unmentioned concepts (e.g., allusions, metaphors), errors (in pronunciation or content), and other irregularities, present NLP systems can have difficulties in identifying action items. Some NLP systems resolve these difficulties by requiring a speaker to utter trigger phrases or other exact wording to signal when a term of interest is to be uttered and how that term is to be interpreted. However, forcing a user to break the flow of a conversation to use various trigger words (and to avoid using those trigger words otherwise), negatively affects the user's ability to converse freely, and may still result in errors if the trigger phrase is not accurately identified. Stated differently, the use of trigger words results in sections of structured language in an otherwise (and preferably) unstructured human language conversation. The present disclosure therefore provides improvements to the NLP systems that improve MLMs via User Interfaces (UIs) that expose at least some the operation of the MLMs to allow the user to converse freely, gain greater trust in the output of the MLMs, and simplify edits to the underlying MLMs, among other benefits.

As the human users interact via the UI with a transcript and the action items (and other extracted elements) identified from the conversation, the UI exposes some or all of the operations of the MLM to the users. By exposing at least some of the operations of the MLMs, the UI provides the users with the opportunity to provide edits and more-relevant feedback to the outputs of the MLMs. Accordingly, the UI gives the users greater control over retraining or updating MLMs for specific use cases. This greater level of control, in turn, provides greater confidence in the accuracy of the MLMs and NLP systems, and thus can expand the functionalities for using the data output by the MLMs and NLP systems or reduce the need for a human user to confirm the outputs of the MLMs and NLP systems. However, in scenarios where the MLMs and NLP systems are still monitored by a human user, or the human user otherwise interacts with or edits the outputs of the MLMs and NLP systems, the UI provides a faster and more convenient way to perform those interactions and edits than previous UIs. Accordingly, the present disclosure is generally directed to increasing and improving the functionality, efficiency, and usability of the underlying computing systems and MLMs via the various methods and apparatuses described herein via an improved UI.

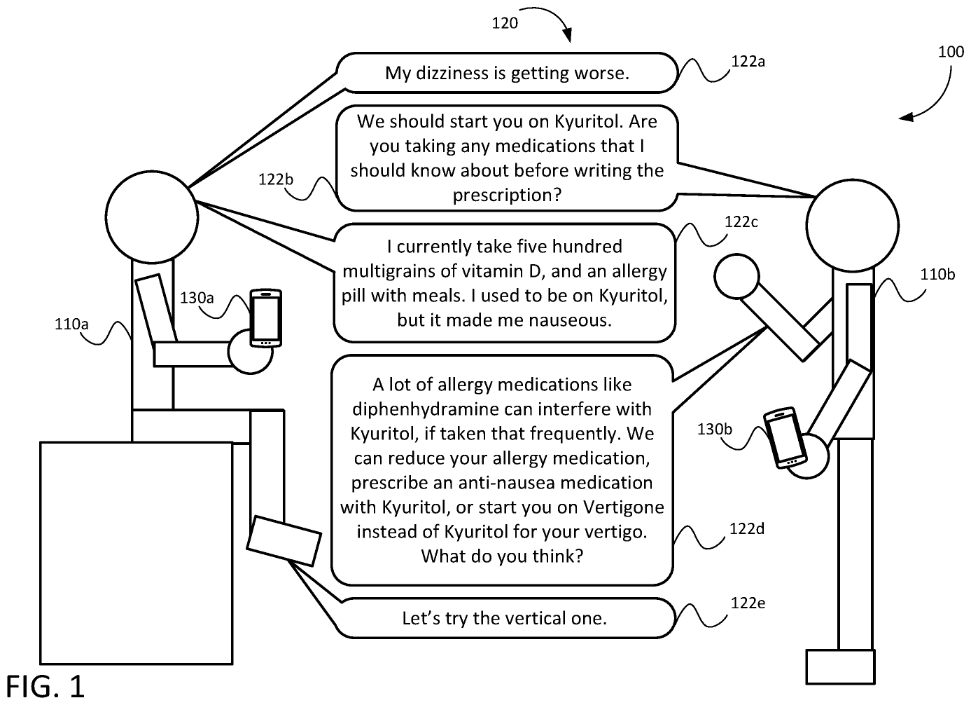

FIG. 1 illustrates an example environment 100 in which a conversation is taking place, according to embodiments of the present disclosure. As shown in FIG. 1, a first party 110a (generally or collectively, party 110) is holding a conversation 120 with a second party 110b. The conversation 120 is spoken aloud and includes several utterances 122a-e (generally or collectively, utterances 122) spoken by the first party 110a and by the second party 110b in relation to a healthcare visit. As shown in the example scenario, the first party 110a is a patient and the second party 110b is a caregiver (e.g., a doctor, nurse, nurse practitioner, physician's assistant, etc.). Although two parties 110 are shown in FIG. 1, in various embodiments, more than two parties 110 may contribute to the conversation 120 or may be present in the environment 100 and not contribute to the conversation 120 (e.g., by not providing utterances 122).

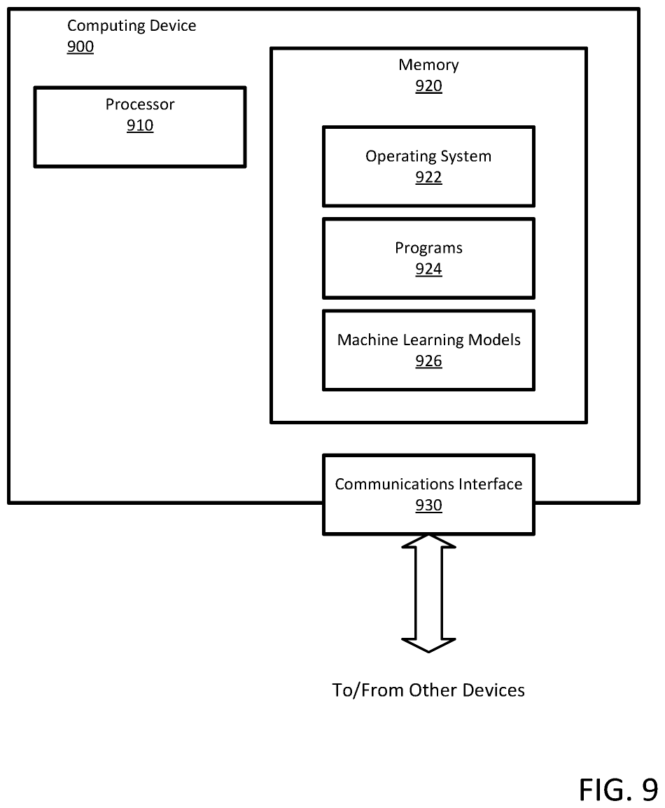

One or more recording devices 130a-b (generally or collectively, recording device 130) are included in the environment 100 to record the conversation 120. In various embodiments, the recording devices 130 may be any device (e.g., such as the computing device 900 described in relation to FIG. 9) that is capable of recording the audio of the conversation, which may include cellphones, dictation devices, laptops, tablets, personal assistant devices, or the like. In various embodiments, the recording devices 130 may transmit the conversation 120 for processing to a remote service (e.g., via a telephone or data network), locally store or cache the recording of the conversation 120 for later processing (locally or remotely), or combinations thereof. In various embodiments, the recording device 130 may pre-process the recording of the conversation 120 to remove or filter out environmental noise, compress the audio, remove undesired sections of the conversation (e.g., silences or user-indicated portions to remove), which may reduce data transmission loads or otherwise increase the speed of transmission of the conversation 120 over a network.

Although FIG. 1 shows two recording devices 130 in the environment 100, where each recording device 130 is associated with one party 110, the present disclosure contemplates other embodiments that may include more or fewer recording devices 130 with different associations to the various parties 110 in the environment 100. For example, a recording device 130 may be associated with the environment 100 (e.g., a recording device 130 for a given room) instead of a party 110, or may be associated with parties 110 who are not participating in the conversation 120, but are present in the environment 100. Additionally, although the environment 100 is shown as a room in which both parties 110 are co-located, in various embodiments, the environment 100 may be a virtual environment or two distant spaces that at linked via teleconference software, a telephone call, or other situation where the parties 110 are not co-located, but are linked technologically to hold the conversation 120.

Recording and transcribing conversations 120 related to healthcare, technology, academia, or various other esoteric topics can be particularly challenging for NLP systems due to the low number of example utterances 122 that include related terms, the inclusion of jargon and shorthand used in the particular domain, the similarities in phonetics of markedly different terms within the domain (e.g., lactase vs. lactose), similar terms having certain meanings inside of the domain that are different from or more specific than the meanings used outside of the domain, mispronunciation or misuse of domain terms by non-experts speaking to domain experts, and other challenges.

One such challenge is that different parties 110 to the conversation 120 may have different levels of experience in the use of the terms used in the conversation 120 or the pronunciation of those terms. For example, an experienced mechanic may refer to a component of an engine by part number, by a nickname, or the specific technical term, while an inexperienced mechanic (or the owner) may refer to the same component via a placeholder (e.g., “the part”), an incorrect term, or an unusual pronunciation (e.g., placing emphasis on the wrong syllable). In another example, a teacher may record a conversation with a student, where the teacher corrects the student's use of various terms or pronunciation, and the conversation 120 includes the misused terminologies, despite both the student and teacher attempting to refer to the same concept. Distinguishing which party 110 is “correct” and that both parties 110 are attempting to refer to the same concept within the domain despite using different wording or pronunciation, can therefore prove challenging for NLP systems.

As illustrated, the conversation 120 includes an exchange between a patient and a caregiver related to the medications that the patient should be prescribed to treat an underlying condition as one example of an esoteric conversation 120 occurring in a healthcare setting. FIG. 1 illustrates the conversation 120 using the intended contents of the utterances 122 from the perspectives of the speakers of those utterances 122, which may include errors made by the speaker. The examples given elsewhere in the present disclosure may build upon the example given in FIG. 1 to variously include misidentified versions of the contents or corrected versions of the contents.

For example, when an NLP system erroneously identifies spoken term A (e.g., the NLP system identified an utterance of be “taste taker”), a user or correction program, may correct the transcription to instead display term B (e.g., changing “taste taker” to “pacemaker” as intended in the utterance). In another example, when a party 110 intended to say term A, and was identified as saying term A, but the correct term is term B, the NLP system can substitutes term B for term A in the transcript.

What term is “correct” may vary based on the level of experience of the party, so that the NLP system may substitute synonymous terms as being more “correct” for the user's context. For example, when a doctor states correctly the chemical name for the allergy medication “diphenhydramine”, the NLP system can “correct” the transcript to read or include additional definitions to state “your allergy medication”. Similarly, various jargon or shorthand phrases may be removed for the more-accessible versions of those phrases in the transcript. Additionally or alternatively, if the party 110 is identified as attempting to say (and mispronouncing) a difficult to pronounce term, such as a chemical name for the allergy medication “diphenhydramine”, (e.g., as “DIFF-enhy-DRAY-MINE” rather than “di-FEN-hye-DRA-meen”), the NLP system can correct the transcript to remove any misidentified terms based on the mispronounced term and substitute in the correct difficult-to-pronounce term.

As intended by the participants of the example conversation 120, the first utterance 122a from the patient includes spoken contents of “my dizziness is getting worse”, to which the caregiver replies in the second utterance 122b “We should start you on Kyuritol. Are you taking any medications that I should know about before writing the prescription?”. The patient replies in the third utterance 122c that “I currently take five hundred multigrains of vitamin D, and an allergy pill with meals. I used to be on Kyuritol, but it made me nauseous.” The caregiver responds in the fourth utterance 122d with “a lot of allergy medications like diphenhydramine can interfere with Kyuritol, if taken that frequently. We can reduce your allergy medication, prescribe an anti-nausea medication with Kyuritol, or start you on Vertigone instead of Kyuritol for your vertigo. What do you think?”. The conversation 120 concludes with the fifth utterance 122e from the patient of “let's try the vertical one.”

Using the illustrated conversation 120 as an example, the patient provided several utterances 122 with misspoken terminology (e.g., “multigrains” instead of “milligrams”, “vertical” instead of “Vertigone” or “vertigo”) that the caregiver did not follow up on (e.g., no question requesting clarification was spoken), as the intended meaning of the utterances 122 was likely clear in context to the caregiver. However, the NLP system may accurately transcribe these misstatements, which can lead to confusion or misidentification of the features of the conversation 120 by a MLM or human user that later reviews the transcript. When later reviewing the transcript, the context may have to be reestablished before the intended meaning of the misspoken utterances can be made clear, thus causing human frustration or errors in analysis systems unless additional time to read and analyze the transcript is expended.

Additionally or alternatively, the inclusion of terms unfamiliar to a party 110 in the conversation 120, even if provided accurately in a later transcript, may lead to confusion or misidentification of the conversation 120 by a MLM or human user. For example, the caregiver mentioned “diphenhydramine”, which may be an unfamiliar term to the patient, despite referring to a popular antihistamine and allergy medication, and the caregiver uses the more scientific-sounding term “vertigo” to refer to condition indicated by the symptom of “dizziness” spoken by the patient, which may have been clear in context at the time of the conversation 120 or glossed over during the conversation 120, but are deserving of follow-up when reviewing the transcript.

The present disclosure therefore provides for UIs that allow users to be able to easily interact with the transcripts to expose various processes of the NLP systems and MLMs that produced and interacted with the conversation 120 and transcripts thereof. A user is thereby provided with an improved experience in examining the transcript and modifying the underlying NLP systems and MLMs to provide more accurate and better trusted analysis results in the future.

Although the present disclosure primarily uses the example conversation related to a healthcare visit shown in FIG. 1 as a basis for the examples discussed in the other Figures, the present disclosure may be used for the provision and manipulation of interactive data gleaned from transcripts of conversations related to various topics outside of the healthcare space or between different parties within the healthcare space. Accordingly, the environment 100 and conversation 120 shown and discussed in relation to FIG. 1 are provided as a non-limiting example; other conversations in other settings (e.g., equipment maintenance, education, law, agriculture, etc.) and between other persons (e.g., a first caregiver and a second caregiver, a guardian and a caregiver, a guardian and a patient, etc.) are contemplated by the present disclosure.

Additionally, although the example conversations and analyzed terms discussed herein are primarily provided in English, the present disclosure may be applied for transcribing a variety of languages with different vocabularies, grammatical rules, word-formation rules, and use of tone to convey complex semantic meanings and relationships between words.

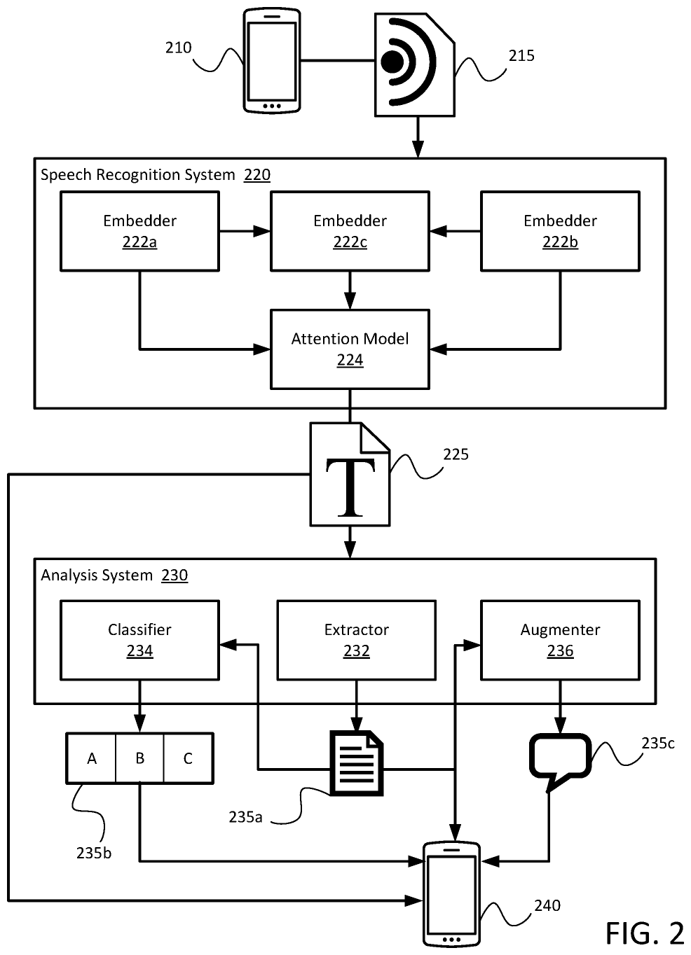

FIG. 2 illustrates a computing environment 200, according to embodiments of the present disclosure. The computing environment 200 may represent a distributed computing environment that includes multiple computers, such as the computing device 900 discussed in relation to FIG. 9, interacting to provide different elements of the computing environment 200 or may include a single computer that locally provides the different elements of the computing environment 200. Accordingly, some or all of the elements illustrated with a single reference number or object in FIG. 2 may include several instances of that element, and individual elements illustrated with one reference number or object may be performed partially or in parallel by multiple computing devices. These various elements may be provided under the control of one of the participants of the conversation to be analyzed, or may be provided by a third party as part of a “cloud” system or by a service

The computing environment 200 includes an audio provider 210, such as a recording device 130 described in relation to FIG. 1, that provides a recording 215 of a completed conversation or individual utterances of an ongoing conversation to a Speech Recognition (SR) system 220 to identify the various words and intents within the conversation. The SR system 220 provides a transcript 225 of the recording 215 to an analysis system 230 to identify and analyze various aspects of the conversation relevant to the participants. As used herein, the SR system 220 and the analysis system 230 may be jointly referred to as an NLP system.

As received, the recording 215 may include an audio file of the conversation, video data associated with the audio data (e.g., a video recording of the conversation vs. an audio-only recording), as well as various metadata related to the conversation, and may also include video data. For example, a user account associated with the audio provider 210 may serve to identify one or more of the participants in the conversation, or append metadata related to the participants. For example, when a recording 215 is received from an audio provider 210 associated with John Doe, the recording 215 may include metadata that John Doe is a participant in the conversation. The user of the audio provider 210 may also indicate that the conversation took place with Erika Mustermann, (e.g., to provide the identity of another speaker not associated with the audio provider 210), when the conversation took place, whether the conversation is complete or is ongoing, where the conversation took place, what the conversation concerns, or the like.

The SR system 220 receives the recording 215 and processes the recording 215 via various machine learning models to convert the spoken conversation into various words in textual form. The models may be domain specific (e.g., trained on a corpus of words for a particular technical field) or general purpose (e.g., trained on a corpus of words for general speech patterns). In various embodiments, the SR system 220 may use an Embedding from Language Models (ELMo) model or a Bidirectional Encoder Representation from Transformers (BERT) model or other machine learning models to convert the natural language spoken audio into a transcribed version of the audio. In various embodiments, the SR system 220 may use Transformer networks, a Connectionist Temporal Classification (CTC) phoneme based model, a Listen Attend and Spell (LAS) grapheme based model, or any of other models to convert the natural language spoken audio into a transcribed version of the audio. In some embodiments, the analysis system 230 may be a large language model (LLM) such as the Generative Pre-trained Transformer 3 (GPT3).

Converting the spoken utterances to a written transcript not only matches the phonemes to corresponding characters and words, but also uses the syntactical and grammatical relationship between the words to identify a semantic intent of the utterance. The SR system 220 uses this identified semantic intent to select the most correct word in the context of the conversation. For example, the words “there”, “their”, and “they're” all sound identical in most English dialects and accents, but convey different semantic intents, and the SR system 220 selects one of the options for inclusion in the transcript for a given utterance. Accordingly, an attention model 224, is used to provide context of the various different candidate words among each other. The selected attention model 224 can use a Long Short Term Memory (LSTM) architecture to track relevancy of nearby words on the syntactical and grammatical relationships between words at a sentence level or across sentences (e.g., to identify a noun introduced in an earlier utterance related to a pronoun in a later utterance).

The SR system 220 can include one or more embedders 222a-c (generally or collectively embedder 222) to embed further annotations to the transcript 225, such as, for example by including: key term identifiers, timestamps, segment boundaries, speaker identifies, and the like. Each embedder 222 may be a trained MLM to identify various features in the audio recording 215 and/or transcript 225 that are used for further analysis by an attention model 224 or extraction by the analysis system 230.

For example, a first embedder 222a is trained to recognize key terms, and may be provided with a set of words, relations between words, or the like to analyze the transcript 225 for. Key terms may be defined to include various terms (and synonyms) of interest to the users. For example, in a medical domain, the names of various medications, therapies, regimens, syndromes, diseases, symptoms, etc., can be set as key terms. In a maintenance domain, the names of various mechanical or electrical components, assurance tests, completed systems, locational terms, procedures, etc., can be set as key terms. In another example, time based words may be identified as candidate key terms (e.g., Friday, tomorrow, last week). Once recognized in the text of the transcript, a key term embedder 222 may embed a metadata tag to identify the related word or set of words as a key term, which may include tagging pronouns associated with a noun with the same metadata tags as the associated noun.

A second embedder 222b can be used by the SR system 220 to recognize different participants in the conversation. In various embodiments, individual speakers may be distinguished by vocal patterns (e.g., a different fundamental frequency for each speaker's voice), loudness of the utterances (e.g., identifying different locations relative to a recording device), or the like.

In another example, a third embedder 222c is trained to recognize segments within a conversation. In various embodiments, the SR system 220 diarizes the conversation into portions that identify the speaker, and provides punctuation for the resulting sentences (e.g., commas at short pauses, periods at longer pauses, question marks at a longer pause preceded by rising intonation) based on the language being spoken. The third embedder 222c may then add metadata tags for who is speaking a given sentence (as determined by the second embedder 222b) and group one or more portions of the sentence together into segments based on one or more of a shared theme or shared speaker, question breaks in the conversation, time period (e.g., a segment may be between X and Y minutes long before being joined with another segment or broken into multiple segments), or the like.

When using a shared theme to generate segments, the SR system 220 may use some of the key terms identified by a key term embedder 222 via string matching. For each of the detected key terms identifying a theme, the segment identifying embedder 222 selects a set of nearby sentences to group together as a segment. For example, when a first sentence uses a noun, and a second sentence uses a pronoun for that noun, the two sentences may be grouped together as a sentence. In another example, when a first person provides a question, and a second person provides a responsive answer to that question, the question and the answer may be grouped together as a segment. In some embodiments, the SR system 220 may define a segment to include between X and Y sentences, where another key term for another segment (and the proximity to the second key term to the first) may define ab edge between adjacent segments.

Once the SR system 220 generates a transcript 225 of the identified words from the recording 215, the SR system 220 provides the transcript 225 to an analysis system 230 to generate various analysis outputs 235 from the conversation. In various embodiments, the operations of the SR system 220 are separately controlled from the operations of the analyses system 230, and the analysis system 230 may therefore operate on a transcript 225 of a written conversation or a human-generated transcript (e.g., omitting the SR system 220 from the NLP system or substituting a non-MLM system for the SR system 220). The SR system 220 may directly transmit the transcript 225 to the output device 240 (before or after the analysis system 230 has analyzed the transcript 225), or the analysis system 230 may transmit the transcript 225 to the output device 240 on behalf of the SR system 220 once analysis is complete.

The analysis system 230 may use an extractor 232 to generate readouts 235a of the key points to provide human-readable summaries of the interactions between the various identified key terms from the transcript. These summaries include the identified key terms (or related synonyms) and are formatted according to factors for sufficiency, minimality, and naturalness. Sufficiency defines a characteristic for a key point that, if given only the annotated span, a reader should be able to predict the correct classification label for the key point, which encourages longer key points that cover all distinguishing or background information needed to interpret the contents of a key point. Minimality defines a characteristic for a key point that identifies peripheral words which can be replaced with other words without changing the classification label for the key point, which discourages marking entire utterances as needed for the interpretation of a key point. Naturalness defines a characteristic for a key point that, if presented to a human reader should sound like a complete phrases in the language used (or as a meaningful word if the key point has only a single key term) to avoid dropping stop words from within phrases and reduce the cognitive load on the human who uses the NLP system's extraction output.

For example, when presented with a series of sentences from the transcript 225 related to how frequently a user should replace a battery in a device, and what type of battery to use, the extractor 232 may analyze several sentences or segments to identify relevant utterances spoken by more than one person to arrive at a summary. The readout 235a may recite “Replace battery; Every year; Use nine volt alkaline” to provide all or most of the relevant information in a human-readable format that was gathered from a much larger conversation.

A category classifier 234 included in the analysis system 230 may operate in conjunction with the extractor 232 to identify various categories 235b that the readouts 235a belong to. In various embodiments, the categories 235b include several different classifications for different users with different review goals for the same conversation. In various embodiments, the category classifier 234 determines the classification based on one or more context vectors developed via the attention layer 224 of the SR system 220 to identify whether a given segment or portion of the conversation belongs to which category (including a null category) out of a plurality of potential categories that a user can select from the system to classify portions of the conversation into.

The analysis system 230 may include an augmenter 236 that operates in conjunction with the extractor 232 to develop supplemental content 235c to provide with the transcript 225. In various embodiments, the supplemental content 235c can include callouts of pseudo-key terms based on inferred or omitted details from a conversation, hyperlinks between key points and semantically relevant segments of the transcript, links to (or provides the content for) supplemental or definitional information to display with the transcript, calendar integration with extracted terms, or the like.

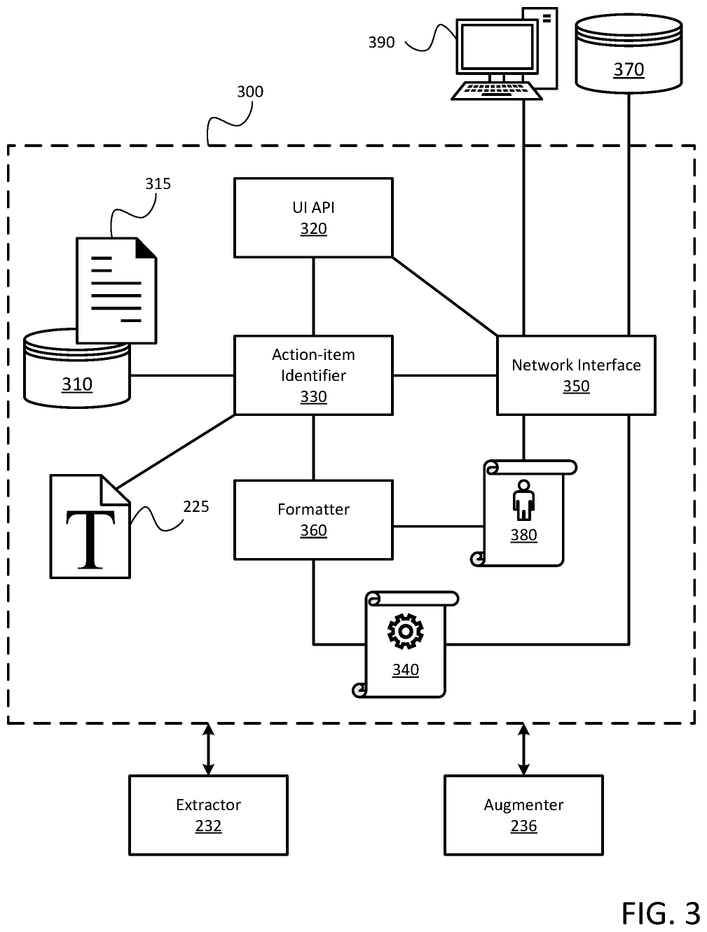

For example, when the extractor 232 identifies terms related to a planned follow up conversation (e.g., “I will call you back in thirty minutes”), the augmenter 236 can generate supplemental content 235c that includes a calendar invitation or reminder in a calendar application associated with one or more of the participants that a call is expected thirty minutes from when the conversation took place. Similarly, if the augmenter 236 identifies terms related to a planned follow up conversation that omits temporal information (e.g., “I will call you back”), the augmenter 236 can generate a pseudo-key term to treat the open-ended follow up as though an actual follow up time had been set (e.g., to follow up within a day or set a reminder to provide a more definite follow up time within a system-defined placeholder amount of time). Additionally or alternatively, the extractor 232 or augmenter 236 can include or use an action-item creator 300 (discussed in greater detail in regard to FIG. 3) that identifies terms from the transcript related to a planned follow up action to the conversation and fills in any details omitted from or left ambiguous in the conversation with supplemental data (e.g., the phone number to call the other party back at) for the conversation.

In various embodiments, when generating supplemental content 235c of a hyperlink between an extracted key point and a segment from the transcript, the augmenter 236 links the most-semantically-relevant segment with the key point, to allow users to navigate to relevant portions of the transcript 225 via the key points. As used herein, the most-semantically-relevant segment refers to the one segment that provides the greatest effect on the category classifier 234 choosing to select one category for the key point, or the one segment that provides the greatest effect on the extractor 232 to identify the key point within the context of the conversation. Stated differently, the most-semantically-relevant segment is the portion of the conversation that has the greatest effect on how the analysis system 230 interprets the meaning and importance of the key point within the conversation.

Additionally, the augmenter 236 may generate or provide supplemental content 235c for defining or explaining various key terms to a reader. For example, links to third-party webpages to explain or provide pictures of various unfamiliar terms, or details recalled from a repository associated with a key term dictionary, can be provided by the augmenter 236 as supplemental content 235c.

The augmenter 236 may format the hyperlink to include the primary target of the linkage (e.g., the most-semantically-relevant segment), various secondary targets to use in updating the linkage based on user feedback (e.g., a next-most-semantically-relevant segment), and various additional effects or content to call based on the formatting guidelines of various programming or markup languages.

Each of the extractor 232, category classifier 234, and the augmenter 236 may be separate MLMs or different layers within one MLM provided by the analysis system 230. Similarly, although illustrated in FIG. 2 with separate modules for an extractor 232, classifier 234, and augmenter 236, in various embodiments, the analysis system 230 may omit one or more of the extractor 232, classifier 234, and augmenter 236 or combine two or more of the extractor 232, classifier 234, and augmenter 236 in a single module. Additionally, the flow of outputs and inputs between the various modules of the analysis system 230 may differ from what is shown in FIG. 2 according to the design of the analysis system 230. When training the one or more MLMs of the analysis system 230, the MLMs may be trained via a first inaccurate supervision technique and subsequently by a second incomplete supervision technique to fine-tune the inaccurate supervision technique and thereby avoid catastrophic forgetting. Additional feedback from the user may be used to provide supervised examples for further training of the MLMs and better weighting of the factors used to identify relevancy of various segments of a conversation to the key points therein, and how those key points are to be categorized for review.

The analysis system 230 provides the analysis outputs 235 to an output device 240 for storage or output to a user. In some embodiments, the output device 240 may be the same or a different device from the audio provider 210. For example, a caregiver may record a conversation via a cellphone as the audio provider 210, and receive and interact with the transcript 225 and analysis outputs 235 of the conversation via the cellphone. In another example, the caregiver may record a conversation via a cellphone as the audio provider 210, and receive and interact with the transcript 225 and analysis outputs 235 of the conversation via a laptop computer.

In various embodiments, the output device 240 is part of a cloud storage or networked device that stores the transcript 225 and analysis outputs 235 for access by other devices that supply matching credentials to allow for access on multiple endpoints.

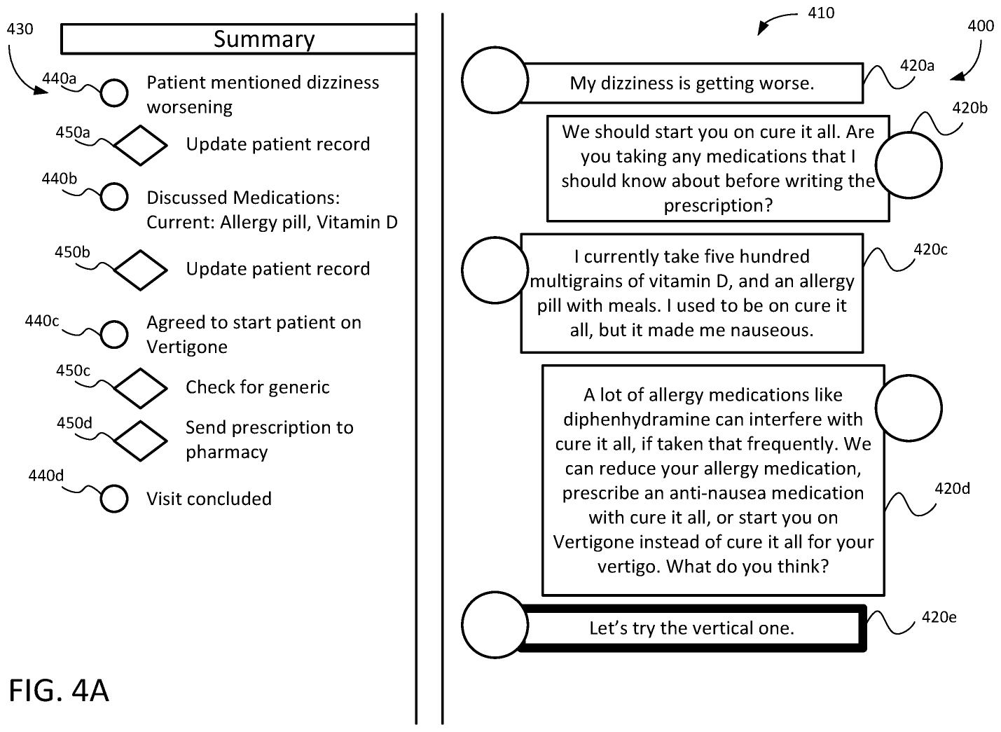

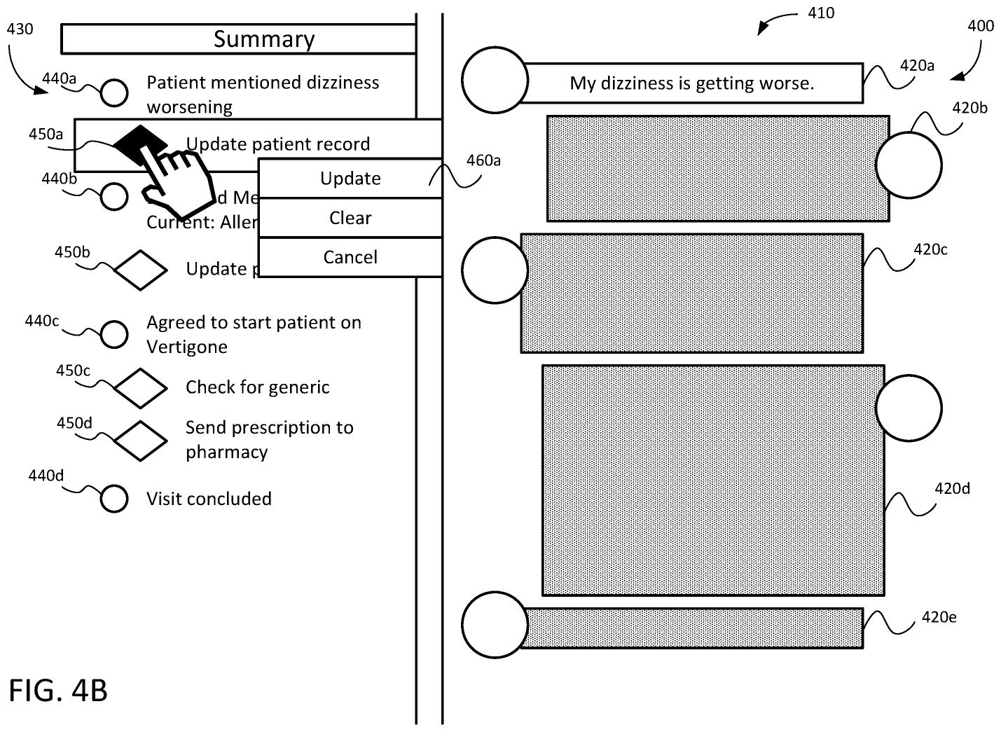

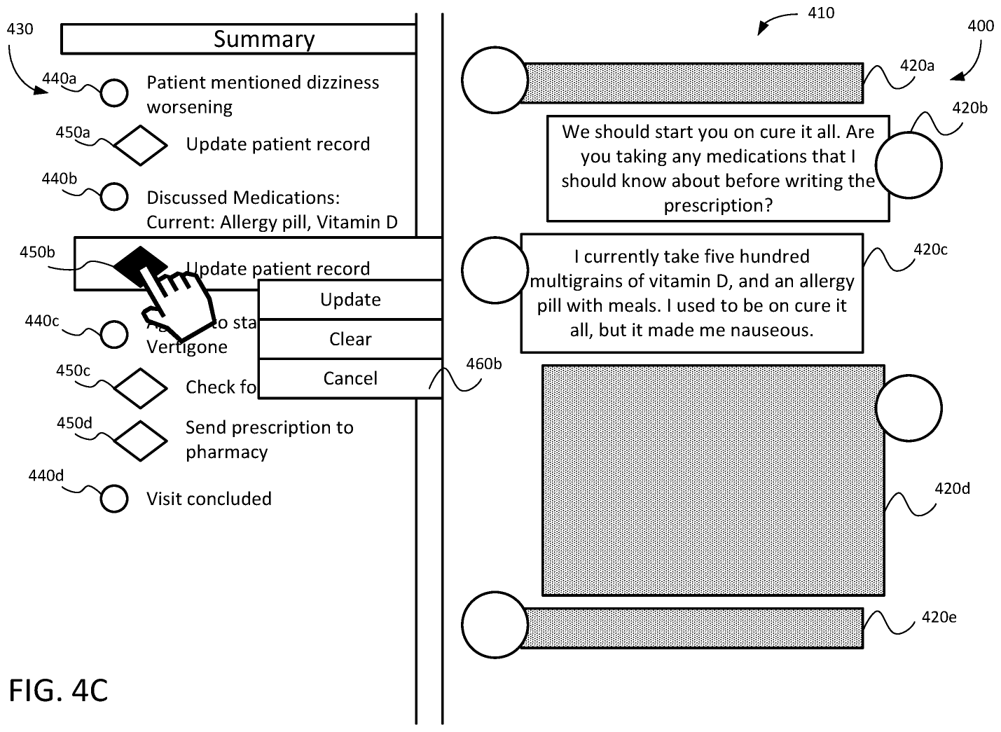

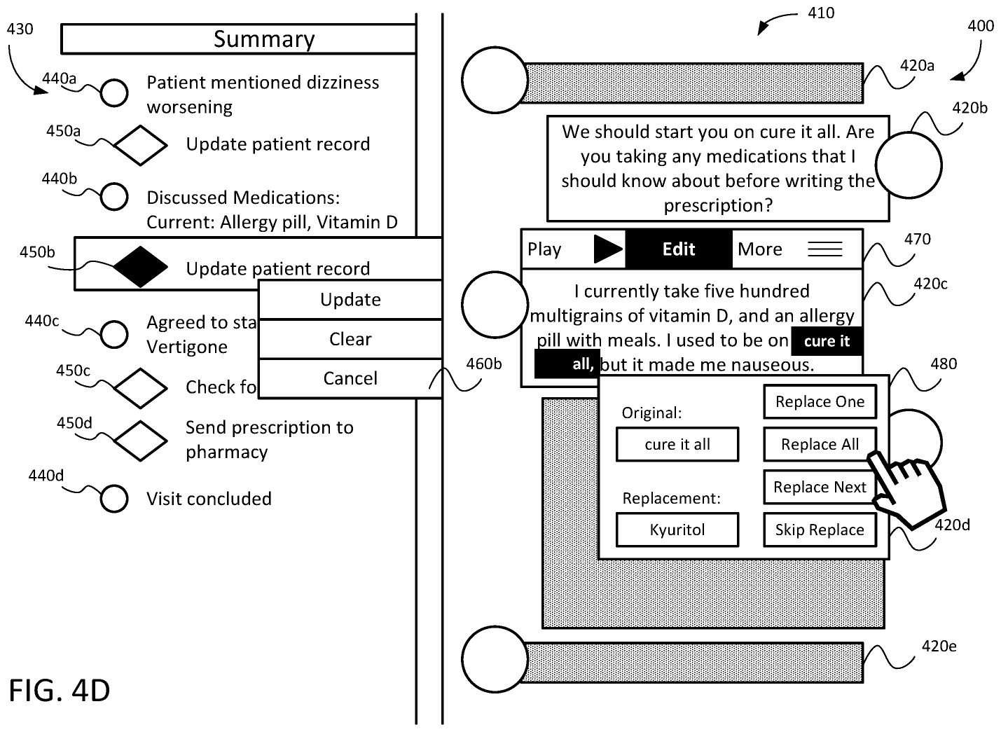

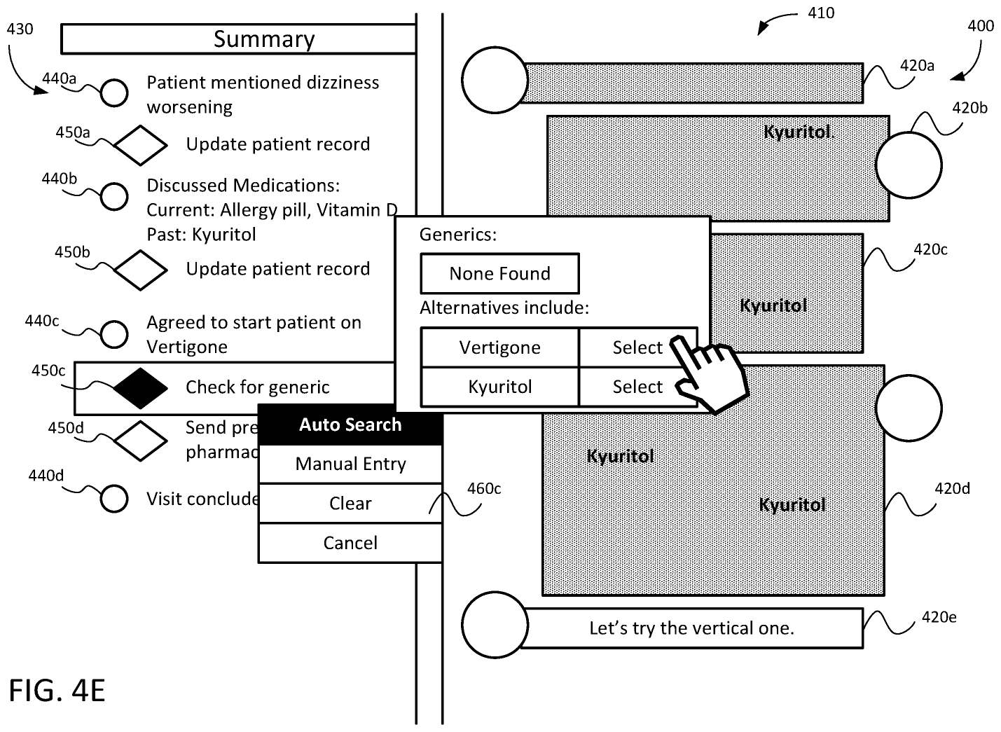

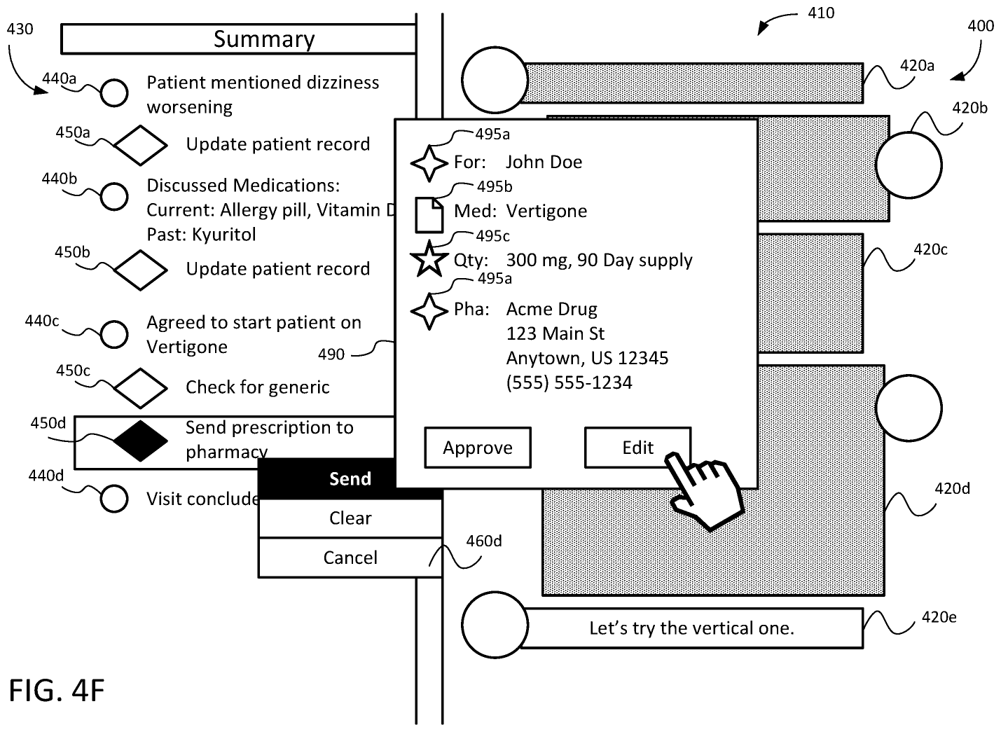

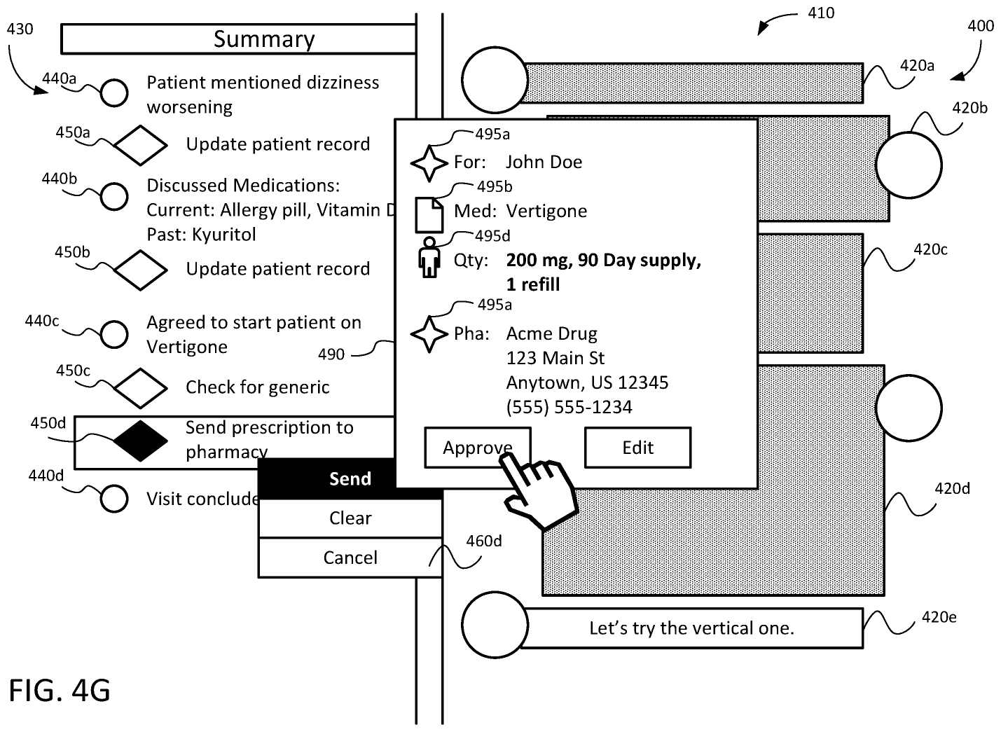

FIG. 3 illustrates an action-item creator 300, according to embodiments of the present disclosure. In various embodiments, the action-item creator 300 is provided as an MLM and the associated modules of computer executable code to identify various action items to follow up on based on a conversation and the information included or omitted therefrom. The action-item creator 300 includes a template database 310 that defines templates 315 for various action items and the data used in fulfilling the action items, a UI Application Program Interface (API) 320 that outputs the action items to a UI (such as those shown in FIGS. 4A-4G), an action-item identifier 330 that requests and receives data from the transcript to identify the action items from the conversation, a network interface 350 that requests and receives data from supplemental data sources 370 and outputs action items to external sources on behalf of the user, and a formatter 360 that can collect the various data and format those data into human readable messages 380 and machine-readable messages 340 respectively for output to human users and automated systems such as supplemental data sources 370 and user systems 390.

In various embodiments, the action-item creator 300 is a module included in, or available for use with, the extractor 232 or augmenter 236, and may use the outputs from the extractor 232 or augmenter 236 as inputs, or provide identified action items as inputs for use by the extractor 232 or augmenter 236. The action-item creator 300 allows the system to generate action items for provision to participants of the conversation (e.g., to the output device 240), messages to non-participant entities (e.g., supplemental data sources 370 or associated output device 240), and handle ambiguity and omission of data from the conversation when generating the action items.

The action-item creator 300 identifies whether elements from the transcript match a template 315 from the template database 310. An action-item identifier 330 may identify the terms and phrases included in the conversation that match one or more templates 315 available from the template database 310 using the context and semantic relevance of each term (e.g., not using trigger words or phrases to activate an action item generator). The action-item identifier 330 may be trained to identify various words associated with known action items (e.g., as set forth in the templates 315), distinguish between phonetically or semantically similar concepts, and identify groupings or pairings of concepts that related to various action items defined by the data fields of one or more templates 315.

For example, a conversation may include two uses of the word “call” where the first occurs in the utterance “it is your call to make which option we choose” and the second occurs in the utterance “I will call you back”, where the first utterance may be an action item for a first person (e.g., to determine which option to choose) and the second utterance may be an action item for a second person (e.g., the speaker) to place a phone call to the first person. Rather than relying on “call” as a trigger word to indicate an action item to place a phone call, which would misidentify the first utterance as being associated with a phone call, the action-item creator 300 uses the action-item identifier 330 to analyze the underlying intent of various segments of the conversation. Accordingly, by using the intent of the utterance, the system is able to analyze natural speech patterns to extract action items from a conversation, and identify supplemental data sources to quickly and accurately cure ambiguities and omissions from the conversation used to complete the generation or execution of the action items.

The action-item identifier 330 may include an LSTM architecture to track relevancy of nearby words based on the syntactical and grammatical relationships between words at a sentence level or across sentences to identify whether the intent of the segment is associated with an action item (e.g., whether a phone call is to be made), the parties associated with an action item (e.g., which entity is to place the phone call, which entity is to receive the phone call), and any additional information related to the action item (e.g., a time to place the phone call, a subject for the phone call, a phone number to use).

The data to include in an action item, and relevant intents behind an action item, may be defined in various templates 315 included in the template database 310. Each template 315 may define a known category of action item and the data used to complete that action item. For example, categories of action items can include “contact other participant,” “contact non-participant party,” “confirm adherence to plan,” or the like that can be further developed based on standard follow-up actions in the user's environment and role in the environment. Various users can develop and specify what data each template 315 specifies to have filled in, when those data need to be provided, and divisions between the various templates 315. For example, a doctor may define templates 315 for referring a patient to another doctor (including data to identify the patient, the condition, and referred-to doctor, etc.), for submitting a prescription to a pharmacy (including data to identify the patient, the medication, the dosage, the amount, etc.), or the like, whereas a mechanic may define templates 315 for performing different procedures on automobiles (including data to identify owner, make of vehicle, service to perform, parts to use, etc.), ordering inventory, and the like, and a music student may define templates 315 specifying the actions to take for different assignments (e.g., including data to identify what songs to practice, specific lessons to monitor during practice, etc.), maintaining an instrument (including data for when to service the instrument), and the like.

Some examples of templates 315 can include record updates, referrals, reminders, queries, confirmations, inventory orders, calendar entries, and the like, each of which may be identified via different contexts and intents from the conversation, and may request different data from the conversation (or a supplemental data source) to complete.

For example, when a segment is identified as potentially matching with templates 315 for starting, stopping, or adjusting a medication, the action-item identifier 330 examines the transcript 225 for various quantities, key terms related to the action to take (e.g., start, begin, stop, cease, adjust, tweak, increase, decrease, put on, take off, etc.) to fill in the details of the template 315. In another example, when a key point is identified as being related to an action item for contacting a party at a later time, the action-item identifier 330 can search the transcript 225 for a preferred medium of communication (e.g., phone, text message, email, post), contact information (e.g., phone number, email address, physical address), time to make contact, and the like.

In various embodiments, after identifying a segment of the transcript 225 that includes data elements relevant to a template 315 for an action item, the action-item identifier 330 analyses other segments of the transcript 225 to gather previously or later mentioned data and to ensure that the action item was not completed during the conversation or otherwise negated. For example, the utterance of “it is your call to make which option we choose” as an action item for a first party to choose an option may be completed with a subsequent utterance from the first part identifying the chosen option. In another example, the utterance of “I will call you back” may be negated with subsequent utterances of “please email me instead” (e.g., replacing the original action item with a different action item) or “no need to call me” (e.g., canceling the original action item).

In various embodiments, the transcript 225 itself may include sufficient data for the action-item creator 300 to fill in the data elements for a given template 315, but the transcript 225 may omit certain data elements, or those data elements may not be initially available in a multipart action item (e.g., a “respond when complete” action item may not have a time element until the other action items are completed). Additionally or alternatively, the data in the transcript 225 may be unreliable or otherwise be of insufficient precision or confidence for use in the template 315. For example, a participant may provide several phone numbers at which they can be reached, while the template 315 calls for one such number, and which phone number to use may be ambiguous without further input. In another example, a participant may have omitted an area code for the phone number, and the action-item creator 300 may therefore have low confidence in the actual phone number.

In an example demonstrating temporally lacking data points, some or all of the data needed to complete various sections of the action item identified by the template 315 may be omitted from the conversation, be unreliable (or mere estimates) when included in the conversation, may not be available until earlier sub-steps have been completed, or are otherwise lacking in the transcript.

For example, when generating action items for installing a catalytic converter based on a conversation between a mechanic and a car owner, the mechanic may need to schedule a repair bay, schedule one or more technicians to perform the work, schedule the use of special equipment, remove the prior catalytic converter, install the new catalytic converter, dispose of the prior catalytic converter, and contact the car owner when complete. In this example, the mechanic is unlikely to commit to using repair bay A or repair bay B during the conversation with the car owner, and although the mechanic may estimate that the repair will be complete by “next Thursday” in the conversation, the repair may be faster or slower than estimated depending on a variety of intervening factors. Accordingly, the action-item creator 300 can expand the information available from the conversation to fill in the data elements with using various external sources, and may leave some elements blank (or later update the values thereof) as time progresses and new data become available.

Additionally, because the transcript 225 of the conversation may include extraneous information, not every word or phrase that could be interpreted as a potential input for a template 315 may be valid for a particular template 315. For example, in a conversation between a teacher and a student, the teacher may tell the student that the practice they put in to preparing song A for a recital was evident in their performance, and the student should practice song B every night until their next lesson, which may result in “practice song B” as an action item for the student from the conversation. However, the information related to practicing song A (in the past), including the identity of the song, a date to practice until, elements of particular note (e.g., tempo, volume, ergonomics) could be valid inputs for an action item for “practice song B”. To distinguish what elements of the conversation to insert into the template, and which to ignore, the action-item creator 300 distinguishes between different parts of the conversation via context in the natural language conversation; avoiding the need to rely on trigger phrases or other explicitly defined data typing operations.

In various examples, the action-item creator 300 can match different templates 315 for different entities. For example, in a medical setting, a treating party (e.g., a doctor, nurse, etc.) and a treated party (e.g., a patient, caregiver, etc.) have different roles, and may respectively have different action items from a conversation about a new medication. The treating party may have an action item of “submit prescription to pharmacy” and the treated party may have an action item of “collect prescription from pharmacy” generated from the same section of the conversation, and would each have different elements extracted from the conversation to fill out these templates 315. Accordingly, a template 315 for submitting a prescription can include data elements for the name of the medication, dosage of the medication, quantity of the medication (or length of prescription), preferred pharmacist, treatment notes, and the like. In contrast, the template 315 for collecting the prescription can include data elements for the preferred pharmacist, medication discount programs, insurance information, and authorized third parties who can collect the prescription. Some of the elements needed to fill out the respective templates may be extracted from the transcript 225, but others may be requested from the user or another supplemental data source 370.

In various examples, the action-item creator 300 can match several templates 315 for one entity and create several actions items for that entity. For example, after a conversation with a car owner about replacing a catalytic converter, a mechanic may have the action item of “install catalytic converter” and “check whether to order additional catalytic converters” from the same section of the conversation.

Once the action-item creator 300 has identified the action items to create for a given entity, the action-item creator 300 attempts to fill in the template 315 with relevant data from the transcript 225. The action-item creator 300 initially uses the action-item identifier 330 to attempt to retrieve the associated data from the transcript 225; however, as the conversation may omit or leave ambiguous various data, the action-item creator 300 may query the user via the UI API 320 to resolve ambiguities or supply missing data, the action-item creator 300 may query a supplemental data source 370 via the network interface 350 to supply missing data, or combinations thereof.

For example, if the conversation resulted in an action item of “make phone call with status update”, the action-item identifier 330 may determine the identity of the entity to whom the phone call is to be placed from the transcript 225 of the conversation, but if the entity's phone number is omitted, the network interface 350 may connect to a database with user details to return a home phone number, a work phone number, and a cell phone number associated with the party to contact. The UI API 320 may then present each of the returned phone numbers to the acting party to select from when making the phone call to provide the other party with a status update.

As used herein, supplemental data refers to data obtained outside of the transcript 225 of the conversation, which may include data provided by a user in response to a query via the UI API 320, data provided by a source under the control of a participant of the conversation (e.g., a database or configuration file with user preferences and user-maintained records) via the network interface 350, and data provided by a source under the control of an entity that was not a party to the conversation (e.g., a directory service, a third-party Electronic Medical Record (EMR) system, an insurance carrier system, a manufacturer's system, a regulator's system, a dictionary or encyclopedia service, or the like) via the network interface 350. A user may specify which systems the network interface 350 is permitted to access to obtain supplemental data from, and a preferred order of attempting to obtain the supplemental data. For example, a doctor may specify that the network interface 350 is to first to attempt to gather supplemental data from a locally hosted EMR system before attempting to request the data from a third-party EMR system or insurer system when generating or updating a local EMR. In another example, the same doctor may specify that the network interface 350 is to first to attempt to gather supplemental data from a third-party EMR system or insurer system (rather than a locally hosted EMR) when submitting a referral to a third party or an insurance authorization request.

In addition to providing the user of the system with outputs related to the action items (e.g., via the UI API 320), the action-item creator 300 can act on behalf of the user to communicate with external systems via a network interface 350. These systems can include the systems used or controlled by the participants of the conversation, systems used or controlled by non-participant entities identified in action items, and system used as supplemental data sources 370. The network interface 350 can transmit a machine-readable message 340 based on the action item and in a specified format from the receiving system via various wired and wireless transmission formats used by different networks (e.g., the Internet, an intranet, a cellular network). The network interface 350 can also receive machine-readable messages 340 including responses to queries and acknowledgment messages that a machine-readable message 340 has been received by the intended recipient.

The formatter 360 converts the natural language transcript 225 (and the values supplied via supplemental data sources 370) into semi-formatted, but still human-readable, action items and into machine-readable formats used by the recipient systems. When converting the portions of the transcript 225 and any supplemental data into action items, the formatter 360 uses the factors of sufficiency, minimality, and naturalness to produce complete, concise, human-readable outputs for presentation to the entity that is to perform the action item. When converting the portions of the transcript 225 and any supplemental data into machine-readable messages 340 for the various systems in communication with the action-item creator 300, the formatter 360 uses the format specified by the receiving system.

For example, when generating a machine-readable message 340 for an EMR database, the formatter 360 generates the machine-readable message 340 as an EMR message. In another example, when generating a referral based on a referral discussion, the formatter 360 can generate a machine-readable message 340 formatted as a referral request according to the format used by an intake system associated with the receiving entity. In another example, the formatter 360 can generate a machine-readable message 340 formatted as a pre-approval request for another action item extracted from the conversation (e.g., to confirm if an owner wants to repair or replace a faulty component identified in an action to “diagnosis issue in component”). In another example, the formatter 360 can generate a machine-readable message 340 formatted as an order form for goods (filled in and supplemented with order details from the conversation), when the action item includes contacting an entity to order components or material.

In various embodiments, the action-item creator 300 may operate on a completed transcript 225 (e.g., after the conversation has concluded) or operate on an in-progress transcript 225 (e.g., while the conversations is ongoing). Accordingly, the action-item creator 300 may, via the UI API 320, generate additional action items while the conversation is ongoing to prompt the participants to discuss additional topics. For example, during an ongoing conversation, the action-item creator 300 may identify an action item to “call other party back” from a partial transcript 225, but receives a reply from a supplemental data source 370 that no phone number is known for the other party (or other request denial), and therefore creates a new human readable message 380 to present an action item to be addressed during the conversation of “ask for phone number”.

The action-item creator 300 uses the network interface 350 to communicate machine-readable messages 340 or the human readable messages 380 to various supplemental data sources 370 and user systems 390, which may represent individual computers or a distributed computing environment that includes multiple computers, such as the computing device 900 discussed in relation to FIG. 9. In various embodiments, the user systems 390 may include the output device 240 discussed in FIG. 2.

The network interface 350 transmits the machine-readable messages 340 that include requests for additional data to various supplemental data sources 370 and user systems 390 and supplies the responsive data to the action-item identifier 330 to fill in any data values initially lacking (e.g., absent from or ambiguous in) in the transcript 225. The network interface 350 may also provide machine-readable messages 340 as automated actions for the action items to assign various tasks or submit data to the various supplemental data sources 370 and user systems 390. Additionally, the network interfaces 350 provides the human-readable messages 380 as UI elements (e.g., via the UI API 320) to the user systems 390 acting as an output device 240, and updates the UI API 320 as the user interacts with the UI elements.

FIGS. 4A-4G illustrate interactions with a UI 400 that displays a transcript and action items identified from a conversation, according to embodiments of the present disclosure. Using the conversation 120 from FIG. 1 as a non-limiting example, the UI 400 illustrated in FIGS. 4A-4G shows a perspective for a caregiver-adapted interface, but in various embodiments, other conversations may relate to different conversational domains taken from different perspectives than those illustrated in the current example.

FIG. 4A illustrates a first state of the UI 400, as may be provided to a user after initial analysis of an audio recording of a conversation by an NLP system. The transcript is shown in a transcript window 410, which includes several segments 420a-420e (generally or collectively, segment 420) identified within the conversation. In various embodiments, the segments 420 may represent speaker turns in the conversation, sentences identified in the conversation, topics identified in the conversation, a given length of time in the conversation (e.g., every X seconds), combinations thereof, and other divisions of the conversation.

Each segment 420 includes a portion of the written text of the transcript, and provides a UI element that allows the user to access the corresponding audio recording, make edits to the transcript, zoom in on the text, and otherwise receive additional detail for the selected portion of the conversation. Although the transcript illustrated in FIGS. 4A-4G includes the entire conversation 120 given as an example in FIG. 1, in various embodiments, the UI 400 may omit portions of the transcript from initial display. For example, the UI 400 may initially display only the segments 420 from which key terms have been identified or action items have been extracted (e.g., to skip introductory remarks or provide a summary), with the non-displayed segments 420 being omitted from display (e.g., positioned “off screen” for later access), shown as thumbnails, etc.

In various embodiments, additional data or metadata related to the segment 420 (e.g., speaker, topic, confidence in written text accurately matching input audio, whether edited by a user) can be presented based on color or shading of the segment 420 or alignment of the segment 420 in the transcript window 410. For example, the first segment 420a, the third segment 420c, and the fifth segment 420e are shown as left-aligned versus the second segment 420b and the fourth segment 420d, which are shown as right-aligned, which indicates different speakers for the differently aligned segments 420. In another example, the fifth segment 420e is displayed with a different shading than the other segments 420, which may indicate that the NLP system is confident that human error is present in the fifth segment 420e, that the NLP system is not confident in the transcribed words matching the spoken utterance, or another aspect of the fifth segment 420e that deserves additional attention from the user.

Depending on the display area available to present the UI 400, the transcript window 410 may include some or all of the segments 420 at a given time. Accordingly, although not illustrated, in various embodiments, the transcript window 410 may include various content controls (e.g., scroll bars, text size controls, etc.) to enable access to more content than can be legibly displayed at one time on the device outputting the UI 400. For example, content controls can allow a user to scroll to currently off-screen elements, zoom in on elements below a size threshold or presented as thumbnails when not selected, or the like.

Outside of the transcript window 410, the UI 400 displays a summary window 430 with one or more summarized key points 440a-d (generally or collectively, key point 440). Some or all of the key points 440 may include various selectable representations 450a-d (generally or collectively, representations 450) of action items extracted from the conversation that are related to the various key points 440. For example, under a first key point 440a of “patient mentioned dizziness worsening”, the UI 400 includes the first representation 450a of “update patient record”. Similarly, under a second key point 440b of “discussed medications: current: allergy pill, vitamin D”, the UI 400 includes the second representation 450b of “update patient record”. The illustrated examples also include a third representation 450c of “check for generic” and a fourth representation 450d of “submit prescription to pharmacy” under the third key point 440c of “agreed to start patient on Vertigone”. However, the key points 440 may omit action items when no follow up action in required (e.g., when the action is completed during the conversation, when no follow up is possible, etc.), such as the illustrated fourth key point 440d that indicates that the visit concluded. Each of the representations 450 provide for the independent display and interaction with the underlying action items identified by the NLP system.

FIG. 4B illustrates selection of the first representation 450a in the UI 400. When a user, via input from one or more of a keyboard, pointing device, voice command, or touch screen, selects a representation 450, the UI 400 may update the display to include various contextual controls 460a-d (generally or collectively, contextual control 460) or highlight related elements in the UI 400 to the selected element. For example, when selecting the first representation 450a, the UI 400 updates to include first contextual controls 460a in association with the first representation 450a to allow editing or further interaction with the underlying action item and elements of the transcript related thereto.

For example, the first contextual controls 460a may offer the user the ability to submit an action item (e.g., to update the patient record on behalf of the user), to clear the action item (e.g., to mark as complete or remove the action item without performing the suggested action), or to cancel (e.g., to dismiss the first contextual controls 460a). As is discussed in greater detail in regard to FIGS. 4E-4F, the contextual controls 460 may include various options and contextual cues based on the context of the representation 450 and underlying action item.

Additionally, the UI 400 adjusts the display of the transcript to highlight the most-semantically-relevant segment 420 to the selected representation 450 for an action item. When highlighting the most-semantically-relevant segment 420, the UI 400 may increase the relative size of the most-semantically-relevant segment 420 to the other segments, but may also change the color, apply an animation effect, scroll which segments 420 are displayed (and where) within the transcript window 410, and combinations thereof to highlight the most-semantically-relevant segment 420 to the selected representation 450. In various embodiments, each representation 450 includes a hyperlink to the corresponding most-semantically-relevant segment 420. The hyperlink includes the location of the most-semantically-relevant segment 420 within the transcript and any effects (e.g., color, animation, resizing, etc.) to apply to the corresponding segment 420 when the representation 450 is selected to thereby highlight it as the most-semantically-relevant segment 420 for the selected representation 450.

Although shown in FIG. 4B with one segment 420 (the first segment 420a) being highlighted in response to receiving a selection of the first representation 450a, in various embodiments, one representation 450 may highlight two or more segments 420 when selected if relevancy carries across segments 420, such as in FIG. 4C. Additionally, multiple representations 450 may indicate a shared (e.g., the same) segment 420 as the respective most-semantically-relevant segment 420. Accordingly, when a user selects different representations 450 associated with a shared segment 420, the UI 400 may apply a different animation effect or new color to the most-semantically-relevant segment 420 to indicate that the later selection resulted in re-highlighting the same segment 420.

By highlighting the segment(s) 420 believed to be the most-semantically-relevant segment(s) 420 to a selected action item, the UI 400 provides the user with an easy approach or manner to navigate to relevant segments 420 of the transcript to review surrounding information related to a core concept that resulted in the identification of the action item. The UI 400 also provides insights into the factors that most influenced the determination that a given segment 420 is the “most-semantically-relevant” segment 420 so that the user can gain confidence in the underlying NLP system's accuracy or correct the misinterpreted segment 420 to thereby have a larger effect on improving the NLP system's accuracy in future analyses.

For example, the conversation presented in the UI 400 may include various ambiguities in interpreting the spoken utterances that the user may wish to fix. These ambiguities may include spoken-word to text conversions (e.g., did the speaker say “sea shells” or “she sells”), semantic relation matching (e.g., is pronoun₁ related to noun₁ or to noun), and relevancy ambiguity (e.g., whether the first discussion of the key point more relevant than the second discussion). By exposing the “most-semantically-relevant” segment 420 to an action item, the user can adjust the linkage between the given segment 420 and the key point to improve later access and review of the transcript, but also provide feedback to the NLP system related to the highest-weighted element from the transcript. Accordingly, the additional functionality provided by the UI 400 improves both the user experience and the computational efficiency and accuracy of the underlying MLM models.

FIGS. 4C and 4D illustrate selection of the second representation 450b in the UI 400 and subsequent editing of the related section of the transcript associated with the second representation 450b. In response to selection of the second representation 450b, the UI 400 updates how the various segments 420 are highlighted. The updated highlighting shows that the most-semantically relevant segments 420 for the discussion of medications key point 440b and associated action item can be found in the second segment 420b and the third segment 420c. The UI 400 also provides a second contextual control 460b in association with the selected second representation 450b. The user is thereby provided with an easy approach or manner to navigate to the relevant segments 420 are review the information contained therein to ensure the accuracy and completion thereof before performing the action item.

For example, as shown in FIG. 4D, the user can see the second key point 440b and identify that the NLP system mistakenly interpreted one or more utterances of “Kyuritol” to be the phonetically similar “cure it all”. The UI 400, in response to the user selecting a third segment 420c, provides segment controls 470 for the user to hear playback of the spoken conversation associated with the written segment 420, edit the written interpretation of the spoken conversation included in the segment 420, and other options. As illustrated, the user has selected to edit the third segment 420c via the segment controls 470, and has selected the text of “cure it all” to be replaced with the text of “Kyuritol”. In various embodiments, the user may supply the replacement text (e.g., via keypad, spoken input, etc.) or is provided with suggested alternatives by the NLP system (e.g., a list of the second-best through nth-best alternatives to replace the original first-best element from the text) to update the text with. The user may replace one, some, or all of the instances of the selected text with the selected replacement text.

In various embodiments, when the user selects a “replace all” option to correct the NLP system's text generation, the correction is sent as feedback to retrain or adjust the MLM used by the NLP system to generate the text (e.g., a training data set). However, when the user selects a “replace one” option to correct a single instance of the text generation, the correction is not sent as feedback to the NLP system, thereby avoiding overfitting the data or unnecessarily retraining the NLP system for unique or atypical terms over more typical terms.

In various embodiments, the user may select a threshold (e.g., at least one, at least two, at least X percent of the occurrences in the transcript) when using the “replace some” option, such that when the threshold number of changes have been made to the transcript (e.g., via a “replace and next” option), the NLP system is provided with positive training examples for the replacement term, and negative training examples for the replacement term when the user chooses not to replace the original term (e.g., via a “skip replace” option). The examples for updating to the new term can also be used in an opposing sense for the maintaining the original term (e.g., negative training examples for the original terms based on the positive training examples for the replacement term, and vice versa). Accordingly, the user is provided with an improved interface to selectively train the NLP system, and thereby customize and improve the underlying NLP systems to the user's use case.

After updating the transcript, as is shown in FIGS. 4E-4G, the user may continue to interact with the UI 400. Although FIGS. 4B-4D illustrate separate interaction with the first representation 450a and the second representation 450b, when two or more representations 450 relate to the same action item (even for different elements of the conversation), the UI 400 may treat completion of one as acceptance for all instances of that action item. For example, the user may separately interact with the representations 450 for “update patient record” to adjust display of the transcript to highlight relevant segments 420, but may submit the “update command” via either set of contextual controls to perform a single update action related to both action items (e.g., updating the patient record to include entries that the “patient mentioned dizziness worsening” and “discussed current medications” via one update to the associate record).

FIG. 4E illustrates selection of the third representation 450c in the UI 400 and automated performance of an action item on behalf of the user. When the user selects a representation 450 associated with an automated action, the system may provide the user with the option (e.g., via a menu of contextual controls 460) for the system to perform the task, or solicit manual entry form the user. For example in FIG. 4E, the third contextual controls 460c associated with the third representation 450c for the action item of “search for generic” may offer a control for authorizing the system to check for generic versions or receive manual entry from the user whether a generic is available.

In some embodiments, in response to receiving authorization from the user to act on their behalf, the system may interface with or query one or more supplemental data sources for related data. The data returned via the automated action may complete the action item, or result in the UI 400 providing a review panel 480 to present the data to the user. In various embodiments, the review panel 480 may include various controls to receive further input from the user. For example in FIG. 4E, the review panel indicates the results of the automated search that no generic versions for Vertigone were found, but that Vertigone and Kyuritol may be used as alternatives for treating the condition indicated in the transcript.

In some embodiments, in response to receiving authorization from the user to act on their behalf, the user's system may interface with an external system of an external entity to submit a machine-readable message. The machine-readable message is intended to complete the interaction from the user's perspective, although the user's system may receive an acknowledgement from the external system. For example, the automated action may transmit an update to a record in a record keeping system, transmit a calendar invitation to a scheduling system, transmit a notice of completion to a system that assigned the action item to the user, or the like.

FIGS. 4F and 4G illustrate selection of the fourth representation 450d in the UI 400 and subsequent editing of the related automated action associated with the fourth representation 450d on behalf of the user. When generating a machine-readable message, the UI 400 may present a human readable element 490 that includes (in a human readable format) the information that will be included in a message to an external system. The human readable element 490 may include various data based on the action item to perform (e.g., as defined by a template 315) and indicate the source of those data by various indicators 495a-d (generally or collectively, indicator 495).

As shown in FIG. 4F, a human readable element 490 is presented when the fourth representation 450d is selected and the user has selected to send the prescription to the pharmacy via the fourth contextual controls 460d. The human readable element 490 is presented as a confirmation before sending a machine-readable message to the system associated with the pharmacy, and includes the various data extracted from the transcript, local systems, and external systems related to the action item. As illustrated, the “for” and “pharmacy” fields are illustrated with a first indicator 495a, indicating that the data in the fields (e.g., the name of the patient and contact information for the patient's pharmacy of record) has been taken from a system associated with the user (e.g., a locally managed EMR system with patient details and preferences). In contrast, the field for the “medicine” is illustrated with a second indicator 495b, indicating that the data (e.g., “Vertigone”—the medication for which the prescription is being submitted) was extracted from the transcript. Similarly, the filed for the “quantity” is illustrated with a third indicator 495c, indicating that the data (e.g., 300 mg, 90 day supply) was received from a supplemental data source 370 that is outside of the user's control (e.g., a pharmacy inventory system, a manufacturer's website, a physician's reference system, an insurance carrier's database of approved medications, etc.).

If the user approves of the extracted data, the user may confirm or approve the system to send a machine-readable message with the data in the appropriate format expected by the recipient entity (e.g., the prescription intake system of Acme Drug in the illustrated example). However, if the user does not approve of the extracted data, the user may manually edit the data, as is shown in FIG. 4G. In FIG. 4G, the user has manually changed the data included in the “quantity” field compared to FIG. 4F, and the UI 400 has updated the third indicator 495c to the fourth indicator 495d to indicate that the user was the source of the supplemental data. In various embodiments, indicators 495 that indicate user entry of data, such as the fourth indicator 495d, may be tied to individual users to record which users made which edits, or may be general purpose indications that some user was the source of the input or edit to the data. Additionally, although not illustrated, when the transcript includes sufficient data to fill in all of the data fields for a certain action item, the UI 400 may display the action item independently or free of indicators 495 for supplemental data sources (e.g., omitting indicators 495 entirely or only displaying indicators 495 for the transcript)

In various embodiments, the various indicators 495 may provide a control element in the UI 400 that allows the user to inspect the source of the data in the associated field. For example, by selecting the first indicator 495a, the user may be provided a pop-up window that displays the user's locally stored EMRs, and allows the user to update the data in the local EMR system for the patient (e.g., changing a preferred pharmacy). In another example, by selecting the second indicator 495b associated with data extracted from the transcript, the UI 400 may adjust display of the segments 420 to display to the user where the data were extracted from. In another example, by selecting the third indicator 495c, the user may be navigated (e.g., via web browser and a hyperlink included in the indicator 495) to a website associated with an external source. Accordingly, the indicators 495 provide the user with additional information about the source of a given data point, and improvements to the ability to investigate how the system determined to use current value for the given data point and to the ability to edit the underlying data or change the source of the data used.

If the source of the supplemental data allows write access from the user, in various embodiments the local edits to data in the UI 400 are propagated to the supplemental data source to implement. In various embodiments, if the source of the supplemental data does not allow write access from the user, the UI 400 may make use of the local edits and inform the data source of the edits (e.g., for tracking when users with disagree or override the supplied data, or for discretionary editing of the data values at the data source).

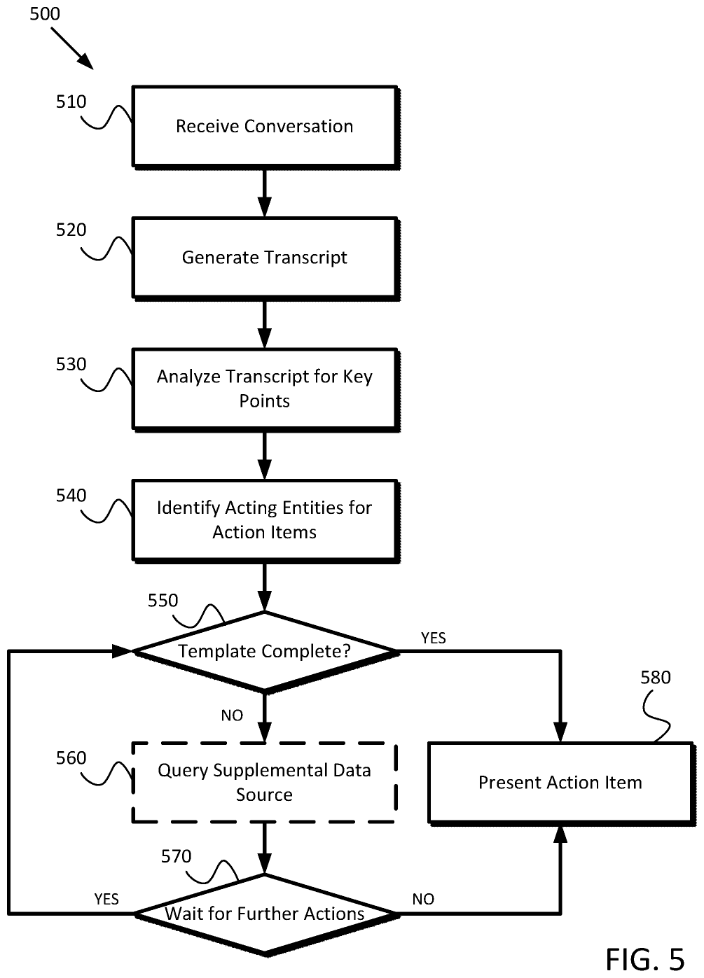

FIG. 5 is a flowchart of a method 500 for presenting action items extracted from a conversation, according to embodiments of the present disclosure. Method 500 begins at block 510, where an NLP system (such as the NLP system including the speech recognition system 220 and analysis system 230 discussed in relation to FIG. 2) receives a conversation that includes utterances spoken by two or more parties. In various embodiments, the recording may be received from a user device associated with one of the parties, and may include various metadata regarding the conversation. Such metadata may include one or more of: the identities of one or more parties, a location where the conversation took place, a time where the conversation took place, a name for the conversation or recording, a user-selected topic of the conversation, whether additional audio sources exist for the same conversation or portions of the conversation (e.g., whether two or more parties are submitting separate recordings of one conversation), etc.

At block 520, a speech recognition system or layer of the NLP system generates a transcript of the conversation included in the recording received at block 510. In various embodiments, the speech recognition system may perform various pre-processing analyses on the audio of the recording to remove background noise or non-speech sounds to aid in analysis of the recording, or may receive the recording having already been processed to emphasize speech. The speech recognition system applies various attention-based models to identify the written words corresponding to the spoken phonemes in the recording to produce a transcript of the conversation. In addition to the phoneme matching, the speech recognition system uses the syntactical and grammatical relationship between the candidate words to identify an intent of the utterance and thereby select words that better match a valid and coherent intent for the natural language speech included in the recording.

In various embodiments, the speech recognition system may clean up verbal miscues, add punctuation to the transcript, and divide the conversation into a plurality of segments to provide additional clarity to readers. For example, the speech recognition system may remove verbal fillers (e.g., “um”, “uh”, etc.), expand shorthand terms, replace or supplement jargon terms with more commonplace synonyms, or the like. The speech recognition system may also add punctuation based on grammatical rules, pauses in the conversation, rising or falling tones in the utterances, or the like. In some embodiments, the speech recognition system uses the various sentences (e.g., identified via the added punctuation) to divide the conversation into segments, but may additionally or alternatively use speaker identities, shared topics/intents, and other features of the conversation to divide the conversation into segments.

At block 530, an analysis system or layer of the NLP system analyzes the transcript of the conversation to identify one or more key terms across the segments of the transcript. In various embodiments, the analysis system identifies key terms based on term-matching the words of the transcript to predefined terms in a key term dictionary or other list. Additionally, because key terms may include multipart phrases, pronouns, or the like, the analysis system analyzes the transcript for nearby elements related to a given key term to provide a fuller meaning for a given term than term matching.

For example, when the word “battery” is identified as a key term and is found in the transcript based on a dictionary match, the analysis system analyzes the sentence that the term is found in, and optionally one or more surrounding sentences before or after the current sentence, to determine whether additional details can better define what the “battery” refers to. The analysis system may thereby determine whether the term “battery” is related to a series of tests, a voltage source, a location, a physical altercation, or a pitching/catching team in baseball, and marks the intended meaning of the key term accordingly. In another example, when the word “appointment” is identified as a key term and is found in one sentence of the transcript, the analysis system may look for related terms (e.g., days, times, relative time terminology) in the current sentence or surrounding sentences to identify whether the appointment refers to the current, past, or future event, and when that event is occurring, has occurred, or will occur.

When identifying the key terms from the transcript, the analysis system may group one or more key terms with supporting words from the transcript to provide a semantically legible summary as a “key point” of that portion of the conversation. For example, instead of merely identifying “battery” and “appointment” as key terms related to the “plan” category, the analysis system may provide a grouped analysis output of “battery replacement appointment next week” to provide a summary that meets the design goals of sufficiency, minimality, and naturalness in presentation of a key point of the conversation. In various embodiments, each key term may be used as a key point if the analysis system cannot identify additional related key terms or supporting words from the transcript to use in conjunction with a lone key term or determines that the key term is sufficient on its own to convey a core concept of the conversation.

At block 540, the NLP system identifies acting entities for action items among the key points identified per block 530. Not all key points extracted from the transcript may be action items, and some key points extracted from the conversation may have multiple (or sub) action items, which may include different action items for different parties based on the same sections of the transcript.

For example, an utterance of “let's work on your technique in playing the diminished c-chord” between a student and a teacher may result in a first action item of “practice chord charts” for the student, and a second action item for “identify songs that use diminished c-chord” for the teacher. Accordingly, when “work on diminished c-chord” is identified as a key point from the transcript, the NLP system can identify two different action items based on what entity is identified as the actor.

In another example, a series of utterances of “my car has trouble stopping” and “let's check if your hydraulic fluid or the brake pads need to be replaced” between a car owner and a mechanic may result in a key point from the conversation of “check brake system”, but may result in two action items for the mechanic (as the actor to check the hydraulic fluid and the brake pads) and no action items for the owner (who has no actions to perform).

In another example, an utterances of “I will generate a ticket with the Internet provider to see if the problem is on their end, and in the meanwhile, I need you to reset your router to see if that solves the connectivity problem” between a technician and a user may result in a first action item for a technician to submit a ticket to the Internet provider and a second action item for the user to reset their router. In some embodiments, the first action item may result in a third action item being assigned to the Internet provider (as an entity that was not part of the conversation) to investigate the user's connection, or the first action item may be omitted, and the third action item is automatically generated and assigned to the Internet provider.

Accordingly, the NLP system identifies the acting entity for the action items (whether a participant or party to the conversation or otherwise) when determining what the action items are. In various embodiments, the acting entity may be identified directly from the transcript, indirectly from the transcript and associated context, or via a supplemental data source. For example, the NLP system can directly identify when a speaker states than a certain party will perform an action (e.g., “I will . . . ”, “you will . . . ”) or infer that a certain party will perform an action based on that party's role when ambiguous language is used (e.g., “we will . . . ” when using the “we” to mean “I” or “you” as a majestic or institutional plural form, using passive voice “the brakes will be checked” that avoids indicating an acting entity, etc.). In another example, the NLP system can identify the identity of an entity named or inferred in the conversation via a supplemental data source, as is described in greater detail with respect block 560, such as when the parties discuss “your child”, “your spouse”, “your parent”, “the supplier”, “my boss”, etc.

At block 550, the NLP system determines whether the template identified for the action item is complete. In various embodiments, the templates may specify one or more acting entities for an action item and various other data points that are used in performing the action item, which may be fully or partially extracted from the transcript. For example, a template for reminding a user of a due date may include fields for the acting entity (e.g., the user) and details for the action to perform (e.g., what assignment is due, when the assignment is due, how to submit the assignment, etc.) that may all have associated values extracted from the transcript, and is therefore complete. In a further example, a template for performing maintenance on a car may include fields for the acting entity (e.g., a mechanic) and details for the action to perform (e.g., identity of the car, maintenance items to perform) that are extracted from the transcript, but lacks certain details (e.g., type of oil to use in oil change, which bay to assign the car to) that may be omitted from the conversation, ambiguous in the conversation, or unknowable at the time of the conversation. As used herein, data that are omitted, ambiguous, or otherwise not identified by the NLP system from the transcript within a confidence threshold may be referred to as “lacking”. For example, a data value for a date and time may be lacking from the transcript if the participants do not discuss a date and time (e.g., omission), discuss multiple dates and times without a clear intent to select one of the dates and times (e.g., ambiguity), the NLP does not identify the selected date and time as being related to the action item, etc.

When the template is complete, method 500 proceeds to block 580. Otherwise, method 500 proceeds to block 560 and block 570 to determine whether additional data should be received before proceeding to block 580.

At block 560, the NLP system queries a supplemental data source for the data missing or left ambiguous in the transcript. Depending on the data missing or left ambiguous in the transcript, and the connections associated with the data, the NLP system may send the query to a user device as the supplemental data source for the user to select from a list of options, provide manual input, or otherwise supply the missing data or clarify the ambiguities. In some embodiments, the NLP system can also query external computing devices either associated with (but not in direct control of the user) or associated with a third party specified by a user to provide the supplemental data. For example, when the action item is to “change oil” in a car, and the conversation does not specify what grade of oil to use, the NLP system may query a maintenance log system controlled by the mechanic to see what grades of oil were previously used or a manufacturer's system (controlled by the manufacturer) to identify what grade of oil the manufacturer recommends for use in the car.

At block 570, the NLP system determines whether to wait for further actions before presenting the action item to the acting entity. In various embodiments, the template may specify what data are required before presenting the action item, or an action item may not be generated until an earlier action item is complete or returns new data. For example, an action item to “install catalytic converter” may not be presented until data are received for the part number for the catalytic converter to install and an action item of “order and receive parts for installation” is completed.

When the NLP system determines to wait for further actions, method 500 may delay for a predefined amount of time, until new data are received from the participants in an ongoing conversation, until new data are received from a supplemental data source, or until a user performs an action (e.g., completing another action item). Method 500 then returns to block 550. Otherwise, method 500 proceeds to block 580.

In various embodiments, method 500 may omit block 560 after some or all instances of checking whether the template is complete at block 550. For example, when the remaining unfilled fields use data that are unknowable at the time of the conversation, method 500 may defer to wait for further actions (per block 570) before querying a supplemental data source (per block 560).

At block 580, the NLP system presents the action item to the acting entity. In various embodiments, the acting entity may be a party to the conversation, but the identified entity to perform the action item can also be a non-participant to the conversation that is identified (per block 540) from the conversation or supplemental data (per block 560). In various embodiments, the NLP system transmits a machine-readable message to the system associated with the acting entity, which can include record updates, referrals, reminders, queries, confirmations, inventory orders, calendar entries, and the like, depending on the action item and type of system used by the acting entity. Method 500 may then conclude.

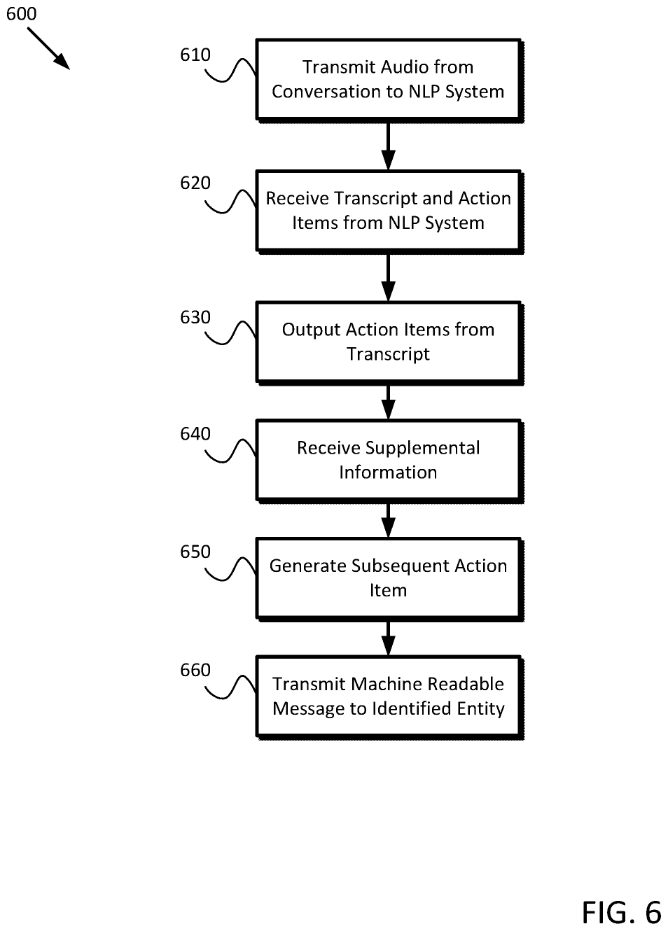

FIG. 6 is a flowchart of a method 600 for using an NLP system to generate a transcript and action items, according to embodiments of the present disclosure. The NLP system discussed in relation to method 600 processes the transcript to extract and perform follow-up actions in which the transcript includes incomplete data for the action items.

Method 600 begins at block 610 where an audio provider transmits audio from a conversation to an NLP system for processing. In various embodiments, the audio provider may include various metadata with the audio, including the location of the conversation, time of the conversation, identities of participants, or the like. The audio includes utterances spoken by at least a first entity and by a second entity who are engaged in a conversation (e.g., the participants). In various embodiments, the audio provider provides the audio of a completed conversation or audio from an ongoing conversation (e.g., as a stream or a batched series of utterances or a given length of time of the conversation) for processing by the NLP to develop a transcript of the conversation and to identify various action items from the conversation for follow up by one or more of the entities. In various embodiments, the NLP system may identify the entity to perform the action item from the participants, or from entities that are not participants in the conversation.

At block 620, an output device (which may be the same device as or a different device than the audio provider) receives a transcript and associated action items from the NLP system generated from the audio provided (per block 610). In various embodiments, the NLP system provides the transcript to multiple output devices, but may provide different action items to the different output devices based on the entity associated with the output device. For example, a first entity may receive action items identified by the NLP system for the first entity to perform, while a second entity may receive different action items that the NLP identified for the second entity to perform.

At block 630, the output device outputs the action items from the transcript to the associated entity. In various embodiments, the output device may display the action items via a UI to an entity of a human user. In some embodiments, the output device outputs the action items as part of a request for supplemental data to clarify ambiguous data, provide omitted data, or provided data that is otherwise not provided in the transcript of the conversation, but is used in an action item. In various embodiments, the request may be a subpart to a multipart action item, or may be a precursor to a subsequent action item, which may be for the same or a different entity to perform.

At block 640, the output device receives supplemental data from the first entity associated with the output device. For example, a user may supply supplemental data that corrects the contents of the action item (e.g., indirectly by correcting the underlying transcript or directly by correcting a representation of the action item). In another example, a user may supply supplemental data that provides values for elements of the action item that were not present in the transcript of the conversation. In another example, the user may supply supplemental data that selects an option presented by the UI, such as an external source to the conversation (e.g., a supplemental data source) that the NLP may query to receive values for elements of the action items that are not present in the transcript of the conversation.

At block 650, the output device (either locally or via the NLP system), generates a subsequent action item based on at least one of the first action item and the supplemental data. In various embodiments, the contents or the acting entity for the subsequent action item are identified from the supplemental data (received per block 640). For example, the supplemental data may supply the identity of the entity to perform the subsequent action item, or a previously lacking value for an element of the subsequent action item that the identified entity is to perform.

In various embodiments, the NLP system may, in response to receiving the supplemental data from the output device, reassign remaining elements of a multipart action item to a different entity (e.g., another party to the conversation or a different entity that was not part of the conversation). For example, after assigning a first action item of “submit prescription” to a doctor, and the doctor providing supplemental data indicating that the prescription has been submitted, the output device may generate a second action item for a patient (who was part of the conversation) to pick up the prescription and a third action item for a pharmacy (that was not part of the conversation) to fill the prescription.

At block 660, the output device (either locally or via the NLP system), transmits a machine-readable message to a computing system associated with the identified entity for the subsequent action item. The machine-readable message is formatted according to the system associated with the identified entity to perform the action item and includes the data elements used by that system to process the action item.

In one example, when the system is a records database, the machine-readable message is formatted to inject data collected during the conversation or during handling the action items into the record associated with one of the participants. For example, when the system is an EMR database, the machine-readable message is formatted as an EMR message with values extracted from the transcript and (optionally) received from supplemental data sources.

In one example, when the identified entity is a service provider that is not one of the participants of the conversation and the action item is a referral to that service provider, the machine-readable message is referral request formatted according to an intake system associated with the service provider, and can include values extracted from the transcript and (optionally) received from supplemental data sources. For example, a conversation between a first physician and a patient can include a discussion or key point of setting up a referral to a second physician (e.g., for a second opinion, for specialist care) who was not a participant of the original conversation, but was mentioned in a referral discussion during the original conversation. In another example, when the identified party is a caretaker (who is not part of the conversation) for a participant, the machine-readable message is formatted as a calendar entry for a caretaker-identified calendaring application.

In one example, when the identified entity is a caretaker or responsible entity for one of the participants in the conversation (e.g., an in-home health assistant, parent, spouse, person holding power of attorney, insurance provider, indemnitor as identified via a record maintained for that participant by another participant), the machine-readable message is a pre-approval request for an action item extracted from the transcript including data values extracted from the transcript and (optionally) received from supplemental data sources. In some embodiments, the pre-approval request can be sent to the responsible entity (who is not a participant in the conversation) while the conversation is ongoing so that the output device can receive a reply from the responsible entity (directly for via the NLP system) approving, denying, or proposing an alternative or new action item.

In one example, when the identified entity is a supplier associated with goods identified in the action item, and the machine-readable message is an order form for the goods that is filled out with data values extracted from the transcript and (optionally) received from supplemental data sources.

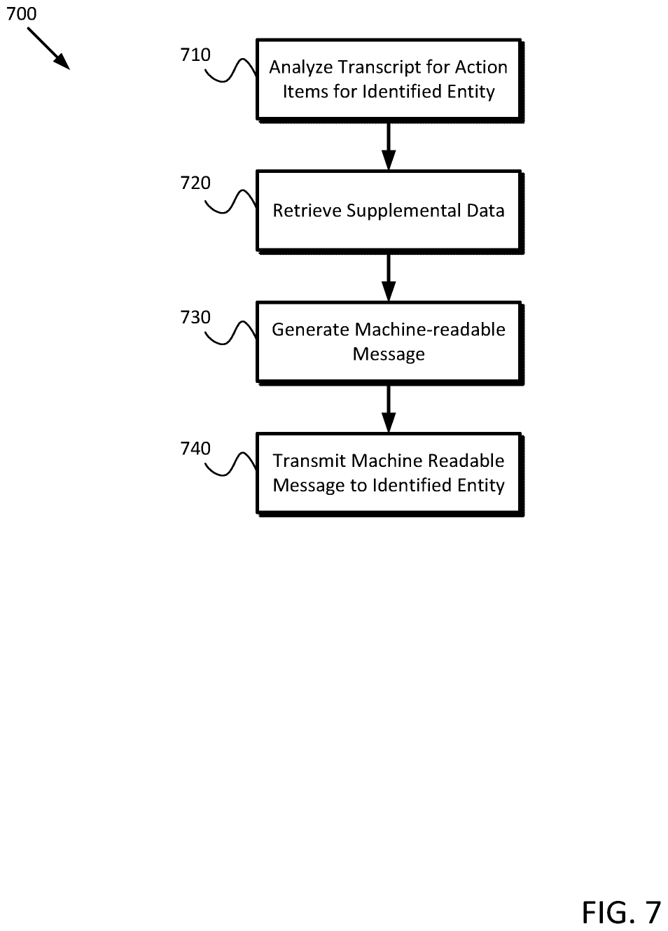

FIG. 7 is a flowchart of a method 700 for automating action item extraction and performance using transcripts of natural language conversation, according to embodiments of the present disclosure. Method 700 begins with block 710, where an NLP system analyzes a transcript of a conversation for action items for identified entries in the transcript. In various embodiments, the NLP system may generate the transcript from audio received from an audio source, or may receive a text transcript of the conversation for further analysis. The NLP system generates a summary of the conversation in a human-readable format, and the summary includes at least one action item that an identified entity is to perform according to the conversation. In various embodiments, the identified entity may be one of the participants in the conversation, or may be an entity that was not a participant in the conversation.

The NLP system identifies the action items as key points within the conversation that include a follow-up task and one or more acting entities to perform the follow-up task. In various embodiments, the NLP system may match the key points to various templates, which allows the NLP system to identify an action item that is missing data from the transcript, and later fill in the missing data with supplemental data. For example, with reference to the conversation from FIG. 1, the patient and doctor have agreed to start a course of Vertigone as a key point of the conversation, which may result in action items for the doctor to submit a prescription, and the patient to pick up the prescription. However, the conversation does not indicate what pharmacy that the doctor will submit the prescription to for the patient to pick up from. Accordingly, the NLP system can identify a “submit prescription” template is partially fillable with data values from the transcript (e.g., who the prescription is for, who is authorizing the prescription, what the prescription is for), but may require additional data to complete (e.g., an identity of a filling pharmacy).

Additionally or alternatively, when the data in the transcript are ambiguous (e.g., the NLP system has two or more candidate values above a confidence threshold, no candidate values above a confidence threshold), the NLP system may refrain from entering a “best guess” for the appropriate data value, and may seek supplemental data to clarify which value or what value to use.

At block 720, the NLP system retrieves supplemental data to fill various data values that are lacking from the transcript. As used herein, data that are lacking include data that are omitted, ambiguous, or otherwise not identified by the NLP system from the transcript within a confidence threshold. For example, a template may specify that a data value for a date and time are included for an action item, but the data may be lacking from the transcript if the participants do not discuss a date and time (e.g., omission), discuss multiple dates and times without a clear intent to select one of the dates and times (e.g., ambiguity), the NLP does not identify the selected date and time as being related to the action item, etc.

In various embodiments, an ambiguous value may be the result of multiple potential values from different utterances in the transcript (e.g., participant 1 says A and participant 2 says B), or may be the result of one utterance or term having a transcription confidence below a threshold value (e.g., participant 1 may have said A or B). When requesting the user to address the ambiguity, the NLP system may present segments of the transcript to the user via a UI to provide additional context to the user to identify the appropriate term to use or to correct/confirm the underlying term choice for an ambiguous term presented in the transcript. In various embodiments, the user's selection is then provided to the NLP for later use as part of a training data set to improve the functionality of the NLP system (e.g., adding the selection, text, and audio to a databased use to provide supervised or semi-supervised training data).

To address the lacking data in the transcript, the NLP system identifies the data values that are lacking for an action item and selects a supplemental data source to provide the omitted value, clarify the ambiguity between utterances, correct an ambiguous term in the transcript with a transcription confidence below a threshold value, or otherwise identify the value to use. In various embodiments, the NLP system may treat one or more of the participants in the conversation as a supplemental data source, and query the participant for the lacking value. In some embodiments, the NLP system may use automated computer systems or entities that were not part of the conversation as supplemental data sources and submit a query on behalf of the user to return the lacking value.

In various embodiments, the template may associate certain values with different supplemental data sources to query first, but may specify one or more secondary data sources (including the user) as fallback supplemental data sources if no automated systems are associated with a certain data field. For example, a user may define the template to query a first local database when a first data field is missing a value and then query the user (or a second database) if the first local database does not provide a responsive data value for the first data field.

At block 730, the NLP system generates a machine-readable message using the format specified by the identified entity to perform the action item, the data values extracted from the transcript of the conversation, and any supplemental data received (per block 720).

In some embodiments, the NLP generates the machine-readable message to complete the action item on behalf of the user. For example, if the action item is to “place order for supplies”, the NLP generates a machine-readable message in the format used by a supplier's ordering system, and automatically fills in the details of the order form using the values extracted from the transcript or supplied from the supplemental data source.

In various embodiments, the NLP system may, in response to receiving the supplemental data from the output device, reassign remaining elements of a multipart action item to a different entity (e.g., another party to the conversation or a different entity that was not part of the conversation). For example, after assigning a first action item of “submit prescription” to a doctor, and the doctor providing supplemental data indicating that the prescription has been submitted, the output device may generate a second action item for a patient (who was part of the conversation) to pick up the prescription and a third action item for a pharmacy (that was not part of the conversation) to fill the prescription.

At block 740, the NLP system transmits the machine-readable message to the identified entity to perform the action item. The machine-readable message is formatted according to the system associated with the identified entity to perform the action item and includes the data elements used by that system to process the action item.

In one example, when the system is a records database, the machine-readable message is formatted to inject data collected during the conversation or during handling the action items into the record associated with one of the participants. For example, when the system is an EMR database, the machine-readable message is formatted as an EMR message with values extracted from the transcript and (optionally) received from supplemental data sources.

In one example, when the identified entity is a service provider not associated with participants of the conversation and the action item is a referral to that service provider, the machine-readable message is referral request formatted according to an intake system associated with the service provider, and can include values extracted from the transcript and (optionally) received from supplemental data sources. For example, a conversation between a first physician and a patient can include a discussion or key point of setting up a referral to a second physician (e.g., for a second opinion, for specialist care) who was not a participant of the original conversation. In another example, when the identified party is a caretaker (who is not part of the conversation) for a participant, the machine-readable message is formatted as a calendar entry for a caretaker-identified calendaring application.

In one example, when the identified entity is a caretaker or responsible entity for one of the participants in the conversation (e.g., an in-home health assistant, parent, spouse, person holding power of attorney, insurance provider, indemnitor as identified via a record maintained for that participant by another participant), the machine-readable message is a pre-approval request for an action item extracted from the transcript including data values extracted from the transcript and (optionally) received from supplemental data sources. In some embodiments, the pre-approval request can be sent to the responsible entity (who is not a participant in the conversation) while the conversation is ongoing so that the output device can receive a reply from the responsible entity (directly for via the NLP system) approving, denying, or proposing an alternative or new action item.

In one example, when the identified entity is a supplier associated with goods identified in the action item, and the machine-readable message is an order form for the goods that is filled out with data values extracted from the transcript and (optionally) received from supplemental data sources.

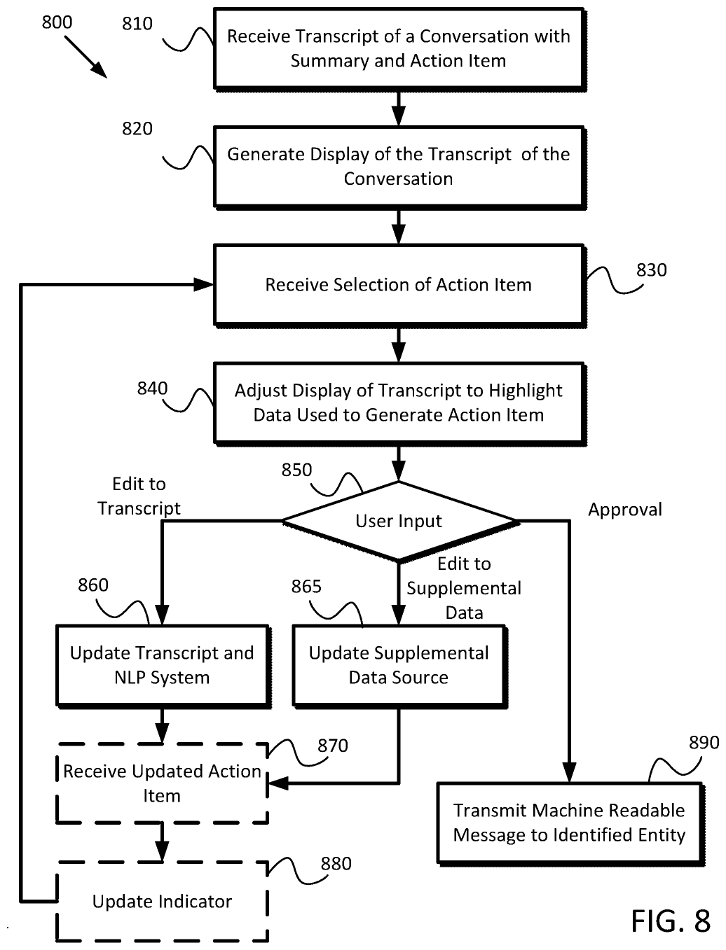

FIG. 8 is a flowchart of a method 800 for displaying transcripts and action items, according to embodiments of the present disclosure. The discussed UIs improve how supplemental data are used in adjusting the transcript and how these supplemental data can be used to improve how the NLP system is used to generate transcripts and extract the action items therefrom.

Method 800 begins at block 810, where an output device receives a transcript of a conversation from an NLP system, the transcript including a summary of the conversation and at least one action item extracted from the conversation for a user of the output device to perform. In various embodiments, the NLP system may generate the transcript from audio received from an audio source (which may include the output device), or may receive a text transcript of the conversation to generate the summary and extract the action items. The NLP system identifies the action items as key points within the conversation that include a follow-up task and one or more acting entities, such as the user of the output device, to perform the follow-up task. Because the conversation may include two or more parties, and reference entities that are not part of the conversation to perform various action items, the NLP system may generate different action items for different entities using the same segments of the transcript.

At block 820, the output device generates a display of the transcript of the conversation including the summary and representations of the one or more action items intended for the user of the output device to perform. The representations of the action items may allow the user to interact with the action items, the data sources for the values used to fill in the action items (including the transcript and supplemental data sources), and the NLP system that produced the action items from the transcript. Example UIs and interactions therewith are discussed in greater detail in regard to FIGS. 4A-4G.

At block 830, the output device receives a selection of a representation of an action item from the user. In various embodiments, the user may make a selection via mouse, keyboard, voice command, touch screen input, or the like.

At block 840, in response to receiving the selection of the representation of the action item (per block 830), the output device adjusts the display of the transcript to highlight the data used to generate the action item. For example, the UI may scroll to, highlight, increase the font size, or otherwise draw attention to the segments of the transcript from which the data used to generate the action item were extracted. Similarly, the UI may scroll away from, deemphasize, decrease the font size, or otherwise draw attention away from the segments of the transcript that are unrelated or provided no data to generate the action item.

By directing the user's attention to the portions of the transcript that are more relevant to generating the action item, the UI provides the user with easier access to the segments to confirm that the NLP system generated accurate action items or to make edits to the action item or transcript to correct inaccuracies from the NLP system. Additionally, because some of the data used to fill in the action items may be received from sources other than the transcript (e.g., supplemental data sources), the UI provides indicators for the supplemental data, to allow the user to verify the source of the data or make corrections to the supplemental data used in the action item, and (optionally) inform the supplemental data source of the edit.

At block 850, the output device receives input from the user. When the user input includes an edit to a segment of the transcript associated with an action item, method 800 proceeds to block 860. When the user input includes an edit to the supplemental data associated with an action item, method 800 proceeds to block 865. When the user input includes approval of the action item, method 800 proceeds to block 890.

At block 860, in response to making an edit to the transcript, the output device updates the transcript as displayed in the UI and updates the NLP system with the change to the transcript for future provision. In various embodiments, the UI can indicate the edited text using a different color, size, typeface, font effect, and combinations thereof relative to the unedited text. Once updated, the NLP system may provide the edited transcript to the editing user when requested again, and may optionally provide a first user's edits to other users.

In various embodiments, the user may make corrections to the transcript and indicate whether the correction is to be provided as a training example for updating how the NLP system transcribes similar audio in the future. For example, the user may indicate that a change to one instance (or another number under an update threshold) of a transcribed term is minor enough that the NLP system should continue to primarily use the original term when transcribing a similar utterance in the future. Stated differently, the user may determine that the transcript should be corrected, but that the NLP system should not be updated to take this correction into account when handling future transcriptions of similar utterances. For example, the user may note that: the speaker has a pronounced accent, misspoke, is using an unusual pronunciation, or is otherwise not providing a representative sample utterance for the term; that the original term is more frequently the correct term than the updated term in similar contexts; that the updated term is an unusual or atypical term that can be confused with more usual or typical terms; or similar features that do not merit (in the user's opinion) updating how the NLP system should handle transcription in the future. Accordingly, the user may indicate via the UI whether various updates to terms in the transcript are to be added to a training data set for retraining the NLP system.

At block 865, in response to making an edit to supplemental data not found in the transcript (but that are used in an action item), the output device optionally sends the update to the supplemental data source.

In various embodiments, the update to the supplemental data may be to a supplemental data source to which the user has write access to. Accordingly, the supplemental data source may receive the edit and implement the edit. For example, when the supplemental data source is a record database used by the user to supplement details of the conversation, and the user identifies data to update (e.g., a new address of the other participant), the edit to the supplemental data in the action item (e.g., updating the address information) is implemented locally in the action item and provided to the record database (e.g., replacing the prior address) for future recall or use.

In some embodiments, the update to the supplemental data may be to a supplemental data source that the user does not have write access to (e.g., read only access). Accordingly, the output device may implement the edit locally to the action item without informing the supplemental data source of the edit, or may inform the supplemental data source of the edit for discretionary implementation. For example, when the supplemental data source is a record database used by the user to supplement details of the conversation, but that requires supervisory review before entering edits, and the user identifies data to update (e.g., a new address of the other participant), the edit to the supplemental data in the action item (e.g., updating the address information) is implemented locally in the action item and provided to the record database for later review and approval or rejection for whether to replace the prior value in the supplemental data source.

At block 870, the output device optionally receives a replacement or updated action item from the NLP system based on the edit to the transcript or supplemental data.

In various embodiments, when the NLP system receives the edit to the transcript from the output device, the change in the transcript may affect what action items are identified in the transcript, or the values used in an existing action item. Accordingly, the NLP system may reanalyze the edited segment of the transcript to determine whether to change a value in an existing action item based on the edit, or produce a new action item that replaces the previous action item. However, not all edits to the transcript may affect the action items.

In an example transcribed conversation where the initial transcription indicates that one party stated “we need to work on your finger ring when playing the diminished the E-chord”, which led to the action item of “practice diminished E-chord”, the user may update the transcription to state “we need to work on your fingering when playing the diminished D-chord”. In this example, the first edit from “finger ring” to “fingering” may be unrelated to the action item, and may be made to the transcript without affecting the action item. However, the second edit from “the diminished the E-chord” to “the diminished D-chord” affects the action item, which may result in the NLP system generating a new action item to replace the initial action item or updating the initial action item to indicate that one party is to “practice diminished D-chord” rather than “practice diminished E-chord”. Accordingly, the NLP system may update the action item based on the second edit, and provide the updated action item to the output device in response to the edit.

At block 880, the output device optionally updates any indicators in the UI associated with the data sources if the source has changed. For example, when the data source was initially the transcript, but the user edited the transcript, the UI may update the indicator to indicate that the user provided the value, or that an updated version of the transcript was used to generate a new data value. In another example, when the user provides manual input for a value for a data field that was initially provided from a supplemental data source, the UI may update the indicator to indicate that the data was received from the user, rather than an external supplemental data source. In another example, when the user selects a different supplemental data source from a list of available data sources, the indicator may be updated to indicate the association with the new data source.

Method 800 returns to block 830, where the updated transcript is displayed for the user, and any updates to the action item are displayed for the user to review. In some embodiments, when a replacement or updated action item is provided to the user (per block 870), the output device automatically selects the new action item for the user to review.

At block 890, when the user input indicates approval of the action item, the NLP system transmits a machine-readable message to an identified entity to perform the action item with the currently assigned values for the data. The machine-readable message is formatted according to the system associated with the identified entity to perform the action item and includes the data elements used by that system to process the action item.

In one example, when the system is a records database, the machine-readable message is formatted to inject data collected during the conversation or during handling the action items into the record associated with one of the participants. For example, when the system is an EMR database, the machine-readable message is formatted as an EMR message with values extracted from the transcript and (optionally) received from supplemental data sources.

In one example, when the identified entity is a service provider not associated with participants of the conversation and the action item is a referral to that service provider, the machine-readable message is referral request formatted according to an intake system associated with the service provider, and can include values extracted from the transcript and (optionally) received from supplemental data sources. For example, a conversation between a first physician and a patient can include a discussion or key point of setting up a referral to a second physician (e.g., for a second opinion, for specialist care) who was not a participant of the original conversation. In another example, when the identified party is a caretaker (who is not part of the conversation) for a participant, the machine-readable message is formatted as a calendar entry for a caretaker-identified calendaring application.

In one example, when the identified entity is a caretaker or responsible entity for one of the participants in the conversation (e.g., an in-home health assistant, parent, spouse, person holding power of attorney, insurance provider, indemnitor as identified via a record maintained for that participant by another participant), the machine-readable message is a pre-approval request for an action item extracted from the transcript including data values extracted from the transcript and (optionally) received from supplemental data sources. In some embodiments, the pre-approval request can be sent to the responsible entity (who is not a participant in the conversation) while the conversation is ongoing so that the output device can receive a reply from the responsible entity (directly for via the NLP system) approving, denying, or proposing an alternative or new action item.

In one example, when the identified entity is a supplier associated with goods identified in the action item, and the machine-readable message is an order form for the goods that is filled out with data values extracted from the transcript and (optionally) received from supplemental data sources.

FIG. 9 illustrates physical components of an example computing device 900 according to embodiments of the present disclosure. The computing device 900 may include at least one processor 910, a memory 920, and a communication interface 930. In various embodiments, the physical components may offer virtualized versions thereof, such as when the computing device 900 is part of a cloud infrastructure providing virtual machines (VMs) to perform some or all of the tasks or operations described for the various devices in the present disclosure.

The processor 910 may be any processing unit capable of performing the operations and procedures described in the present disclosure. In various embodiments, the processor 910 can represent a single processor, multiple processors, a processor with multiple cores, and combinations thereof. Additionally, the processor 910 may include various virtual processors used in a virtualization or cloud environment to handle client tasks.

The memory 920 is an apparatus that may be either volatile or non-volatile memory and may include RAM, flash, cache, disk drives, and other computer readable memory storage devices. Although shown as a single entity, the memory 920 may be divided into different memory storage elements such as RAM and one or more hard disk drives. Additionally, the memory 920 may include various virtual memories used in a virtualization or cloud environment to handle client tasks. As used herein, the memory 920 is an example of a device that includes computer-readable storage media, and is not to be interpreted as transmission media or signals per se.

As shown, the memory 920 includes various instructions that are executable by the processor 910 to provide an operating system 922 to manage various operations of the computing device 900 and one or more programs 924 to provide various features to users of the computing device 900, which include one or more of the features and operations described in the present disclosure. One of ordinary skill in the relevant art will recognize that different approaches can be taken in selecting or designing a program 924 to perform the operations described herein, including choice of programming language, the operating system 922 used by the computing device, and the architecture of the processor 910 and memory 920. Accordingly, the person of ordinary skill in the relevant art will be able to select or design an appropriate program 924 based on the details provided in the present disclosure.

Additionally, the memory 920 can include one or more of machine learning models 926 for speech recognition and analysis, as described in the present disclosure. As used herein, the machine learning models 926 may include various algorithms used to provide “artificial intelligence” to the computing device 900, which may include Artificial Neural Networks, decision trees, support vector machines, genetic algorithms, Bayesian networks, or the like. The models may include publically available services (e.g., via an Application Program Interface with the provider) as well as purpose-trained or proprietary services. One of ordinary skill in the relevant art will recognize that different domains may benefit from the use of different machine learning models 926, which may be continuously or periodically trained based on received feedback. Accordingly, the person of ordinary skill in the relevant art will be able to select or design an appropriate machine learning model 926 based on the details provided in the present disclosure.

The communication interface 930 facilitates communications between the computing device 900 and other devices, which may also be computing devices 900 as described in relation to FIG. 9. In various embodiments, the communication interface 930 includes antennas for wireless communications and various wired communication ports. The computing device 900 may also include or be in communication, via the communication interface 930, one or more input devices (e.g., a keyboard, mouse, pen, touch input device, etc.) and one or more output devices (e.g., a display, speakers, a printer, etc.).

Accordingly, the computing device 900 is an example of a system that includes a processor 910 and a memory 920 that includes instructions that (when executed by the processor 910) perform various embodiments of the present disclosure. Similarly, the memory 920 is an apparatus that includes instructions that when executed by a processor 910 perform various embodiments of the present disclosure.

Programming modules, may include routines, programs, components, data structures, and other types of structures that may perform particular tasks or that may implement particular abstract data types. Moreover, embodiments may be practiced with other computer system configurations, including hand-held devices, multiprocessor systems, microprocessor-based or programmable user electronics, minicomputers, mainframe computers, and the like. Embodiments may also be practiced in distributed computing environments where tasks are performed by remote processing devices that are linked through a communications network. In a distributed computing environment, programming modules may be located in both local and remote memory storage devices.

Furthermore, embodiments may be practiced in an electrical circuit comprising discrete electronic elements, packaged or integrated electronic chips containing logic gates, a circuit using a microprocessor, or on a single chip containing electronic elements or microprocessors (e.g., a system-on-a-chip (SoC)). Embodiments may also be practiced using other technologies capable of performing logical operations such as, for example, AND, OR, and NOT, including, but not limited to, mechanical, optical, fluidic, and quantum technologies. In addition, embodiments may be practiced within a general purpose computer or in any other circuits or systems.

Embodiments may be implemented as a computer process (method), a computing system, or as an article of manufacture, such as a computer program product or computer-readable storage medium. The computer program product may be a computer-readable storage medium readable by a computer system and encoding a computer program of instructions for executing a computer process. Accordingly, hardware or software (including firmware, resident software, micro-code, etc.) may provide embodiments discussed herein. Embodiments may take the form of a computer program product on a computer-usable or computer-readable storage medium having computer-usable or computer-readable program code embodied in the medium for use by, or in connection with, an instruction execution system.

Although embodiments have been described as being associated with data stored in memory and other storage mediums, data can also be stored on or read from other types of computer-readable media, such as secondary storage devices, like hard disks, floppy disks, or a CD-ROM, or other forms of RAM or ROM. The term computer-readable storage medium refers only to devices and articles of manufacture that store data or computer-executable instructions readable by a computing device. The term computer-readable storage medium does not include computer-readable transmission media.

Embodiments described in the present disclosure may be used in various distributed computing environments where tasks are performed by remote processing devices that are linked through a communications network.

Embodiments described in the present disclosure may be implemented via local and remote computing and data storage systems. Such memory storage and processing units may be implemented in a computing device. Any suitable combination of hardware, software, or firmware may be used to implement the memory storage and processing unit. For example, the memory storage and processing unit may be implemented with computing device 900 or any other computing devices 922, in combination with computing device 900, wherein functionality may be brought together over a network in a distributed computing environment, for example, an intranet or the Internet, to perform the functions as described herein. The systems, devices, and processors described herein are provided as examples; however, other systems, devices, and processors may comprise the aforementioned memory storage and processing unit, consistent with the described embodiments.

The descriptions and illustrations of one or more embodiments provided in this application are intended to provide a thorough and complete disclosure of the full scope of the subject matter to those of ordinary skill in the relevant art and are not intended to limit or restrict the scope of the subject matter as claimed in any way. The embodiments, examples, and details provided in this disclosure are considered sufficient to convey possession and enable those of ordinary skill in the relevant art to practice the best mode of the claimed subject matter. Descriptions of structures, resources, operations, and acts considered well-known to those of ordinary skill in the relevant art may be brief or omitted to avoid obscuring lesser known or unique aspects of the subject matter of this disclosure. The claimed subject matter should not be construed as being limited to any embodiment, aspect, example, or detail provided in this disclosure unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently.

Having been provided with the description and illustration of the present disclosure, one of ordinary skill in the relevant art may envision variations, modifications, and alternative embodiments falling within the spirit of the broader aspects of the general inventive concept provided in this disclosure that do not depart from the broader scope of the present disclosure.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

The following claims are not intended to be limited to the embodiments shown herein, but are to be accorded the full scope consistent with the language of the claims. Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

1. A method, comprising:
analyzing a transcript of a conversation, by a Natural Language Processing (NLP) system, to generate a summary of the conversation in a human-readable format, the summary including action items associated with an identified entity;
retrieving, by the NLP system from a supplemental data source, supplemental data associated with the action item that are lacking in the transcript;
generating, by the NLP system, a machine-readable message based on the action item and the supplemental data; and
transmitting the machine-readable message to a system associated with the identified entity.

2. The method of claim 1, wherein the identified entity is not a participant in the conversation.

3. The method of claim 2, wherein the system is an Electronic Medical Record (EMR) database associated with the identified entity, and the machine-readable message is formatted as an EMR message.

4. The method of claim 2, further comprising:
identifying a referral discussion in the transcript;
wherein the identified entity is a service provider not associated with participants of the conversation that is identified via at least one of the referral discussion in the transcript and a referral list associated with at least one of the participants of the conversation, wherein the machine-readable message is referral request formatted according to an intake system associated with the service provider.

5. The method of claim 2, wherein the identified entity is a responsible entity associated with a second entity of the conversation via a record maintained by a first entity in the conversation for the second entity, wherein machine-readable message is a pre-approval request for a second action item discussed in the transcript.

6. The method of claim 5, further comprising:
sending the pre-approval request to the responsible entity while the conversation is ongoing;
receiving a reply from the responsible entity denying the pre-approval request; and
generating a third action item while the conversation is ongoing to prompt the first entity to propose an alternative to the second action item.

7. The method of claim 2, wherein the identified entity is a supplier associated with goods identified in the action items, wherein the machine-readable message is an order form for the goods supplemented with order details for a participant of the conversation.

8. The method of claim 2, wherein the identified entity is a caretaker for a participant of the conversation, wherein the caretaker is identified via a patient record for the participant, wherein the machine-readable message is associated with a caretaker-identified calendaring application.

9. The method of claim 1, wherein the supplemental data are requested from a participant of the conversation by the NLP system for at least one of:
clarifying a term in the transcript with a transcription confidence below a threshold value;
supplying a value missing from the transcript for an element of the action items; and
selecting one of a list of ambiguous terms for inclusion in the action item.

10. The method of claim 1, wherein the action items are created by the NLP system based on terminology and context from the transcript.

11-20. (canceled)

21. A system, comprising:
a processor; and
a memory including instructions that when executed by the processor perform operations comprising:
analyzing a transcript of a conversation, by a Natural Language Processing (NLP) system, to generate a summary of the conversation in a human-readable format, the summary including action items associated with an identified entity;
retrieving, by the NLP system from a supplemental data source, supplemental data associated with the action item that are lacking in the transcript;
generating, by the NLP system, a machine-readable message based on the action item and the supplemental data; and
transmitting the machine-readable message to a computing system associated with the identified entity.

22. The system of claim 21, wherein the identified entity is not a participant in the conversation.

23. The system of claim 22, wherein the computing system is an Electronic Medical Record (EMR) database associated with the identified entity, and the machine-readable message is formatted as an EMR message.

24. The system of claim 22, the operations further comprising:
identifying a referral discussion in the transcript;
wherein the identified entity is a service provider not associated with participants of the conversation that is identified via at least one of the referral discussion in the transcript and a referral list associated with at least one of the participants of the conversation, wherein the machine-readable message is referral request formatted according to an intake system associated with the service provider.

25. The system of claim 22, wherein the identified entity is a responsible entity associated with a second entity of the conversation via a record maintained by a first entity in the conversation for the second entity, wherein machine-readable message is a pre-approval request for a second action item discussed in the transcript.

26-40. (canceled)

41. A memory device including instructions that when executed by a processor perform operations comprising:
analyzing a transcript of a conversation, by a Natural Language Processing (NLP) system, to generate a summary of the conversation in a human-readable format, the summary including action items associated with an identified entity;
retrieving, by the NLP system from a supplemental data source, supplemental data associated with the action item that are lacking in the transcript;
generating, by the NLP system, a machine-readable message based on the action item and the supplemental data; and
transmitting the machine-readable message to a computing system associated with the identified entity.

42. The memory device of claim 41, wherein the identified entity is not a participant in the conversation.

43. The memory device of claim 42, wherein the computing system is an Electronic Medical Record (EMR) database associated with the identified entity, and the machine-readable message is formatted as an EMR message.

44. The memory device of claim 42, the operations further comprising:
identifying a referral discussion in the transcript;
wherein the identified entity is a service provider not associated with participants of the conversation that is identified via at least one of the referral discussion in the transcript and a referral list associated with at least one of the participants of the conversation, wherein the machine-readable message is referral request formatted according to an intake system associated with the service provider.

45. The memory device of claim 42, wherein the identified entity is a responsible entity associated with a second entity of the conversation via a record maintained by a first entity in the conversation for the second entity, wherein machine-readable message is a pre-approval request for a second action item discussed in the transcript.

46-60. (canceled)
